(19) United States
(12) Patent Application Publication
(10) Pub. No.: US 2006/0230465 A1
(43) Pub. Date: Oct. 12, 2006

(54) K203 gene and protein

(76) Inventors: Andreas Winterpacht, Erlangen (DE); Gerrit Mohrmann, Hamburg (DE); Jan Georg Hengstler, Leipzig (DE); Hans Will, Hamburg (DE); Thomas Hofmann, Eppelheim (DE)

Correspondence Address: JENKINS, WILSON, TAYLOR & HUNT, P. A., 3100 TOWER BLVD, SUITE 1200, DURHAM, NC, 27707, US

(21) Appl. No.: 11/373,761

(22) Filed: Mar. 10, 2006

Related U.S. Application Data
(63) Continuation of application No. PCT/EP04/10159, filed on Sep. 10, 2004.

(51) Int. Cl. A01K 67/027; C12Q 1/68; G01N 33/574; C07H 21/04; C12P 21/06; A61K 39/395; C07K 14/82; C07K 16/30

(52) U.S. Cl. 800/8; 435/6; 435/7.23; 435/69.1; 435/320.1; 435/325; 514/44; 424/155.1; 530/350; 530/388.8; 536/23.5

(57) ABSTRACT

The present invention is directed to the human and murine K203 protein and gene and to K203 binding compounds. Furthermore, the present invention relates to a pharmaceutical and diagnostic composition for use in the diagnosis and treatment of cancer as well as to a method for the diagnosis of cancer and a method of treating same.

### RELATED APPLICATIONS

This application is a continuation of PCT International Patent Application No. PCT/EP2004/010159, filed Sep. 10, 2004, which claims priority to U.S. Provisional Patent Application No. 60/502,052, filed September 10, 2003, the disclosures of each of which are incorporated herein by reference in their entirety.

The present invention is directed to the human and murine K203 protein and gene and to K203 binding compounds. Furthermore, the present invention relates to a pharmaceutical and diagnostic composition for use in the diagnosis and treatment of cancer as well as to a method for the diagnosis of cancer and a method of treating same.

The present inventor based the invention on the surprising discovery that the expression of K203 is correlated with the prognosis of different cancers, in particular ovarian carcinoma. It turned out that there is a good correlation with a prognosis depending on the remaining tumor tissue and survival time. Therefore, the expression of K203 can be used as a prognostic marker. Additionally, a therapy against cancer may be established based on K203.

On the one hand, ovarian carcinoma is one of the more rare carcinomas of women in contrast to mammary carcinoma or lung carcinoma. On the other hand, ovarian carcinoma has a leading role in death caused by cancer due to its poor chances for healing.

The incidents, i.e. the number of new cases per year, in Northern Europe is 14:100,000 whereas it is only 3:100,000 in Japan. In the Federal Republic of Germany every 66^{th} woman will be afflicted by ovarian carcinoma, and as a comparative example, every 8^{th}-10^{th} woman will be afflicted by mammary carcinoma. Ovarian carcinoma is a disease of the second half of life following the menopause, the peak occurring in the 7^{th} and 8^{th} decade of life.

The causes underlying the disease are up to now unknown, however, the cumulative occurrence in certain families points to a genetic cause.

For the prognosis of the disease, several parameters are of importance, for example the distribution of the disease as well as the stage of tumor, histology and the size of the remaining tumor tissue following surgery.

Patients having a disease limited to the ovaries have a 5-year survival rate of between 70 and 90%. However, if the cancer has already extended to the pelvis, the 5-year survival rate is reduced to 45-47%. If a peritoneal carcinoma and lymph node metastasis is present, the 5-year survival rate is decreased to 17-24%. If remote metastases have already occurred via the blood path, only 5-12% of the patients will survive the next 5 years.

The survival rate is additionally shortened dependently from the remaining tumor tissue following surgery. In this case, the volume of the biggest remaining tissue is of more importance as the number of the remaining tissues in general. Remaining tumor tissue, which is smaller than one cm³ means a 5 year survival rate between 60 and 70%. In about 60-70% of all patients having advanced ovarian carcinoma recidivisms will occur, wherein 90-95% of these recidivisms will occur within the first 5 years. The earlier the recidivism will occur the poorer the prognosis of the patient is. The main death cause of patients having ovarian carcinoma is peritoneal carcinosis.

In the patent literature, there are indications that portions of the sequence of K203 as disclosed herein, have already been published.

In WO 01/57190 (Novel nucleic acids and polypeptides of Hysac Incorporated) about 4,000 nucleic acids are claimed for the diagnosis of diverse diseases. One of the sequences partially matches K203. However, it is not mentioned that the sequence can be used for the function disclosed herein, in particular not in the prognosis of ovarian carcinoma.

WO 02/18632 claims more than 40,000 sequences for diagnostic methods, wherein a precise pattern of methylation has to be considered. However, no mention is made of the carcinomas disclosed herein.

It is an object of the present invention to provide genes and proteins, which can be advantageously used as the base for diagnosis and therapy of several kinds of carcinomas.

This object is solved by the subject-matter of the independent claims. Preferred embodiments are set forth in the dependent claims.

Herein, a novel cDNA from a human fetal cartilage cDNA library (UniGene cluster Hs. 7299) is disclosed. The inventor determined 3.7 kb of the cDNA using RT-PCR in combination with the present UniGene sequence data and predicted exons from the genomic sequence. The determined cDNA covers the complete coding region and includes the putative translation start and stop codon. Furthermore, the complete coding sequence of the orthologues murine gene was isolated which shows 87.3% sequence identity to human. cDNA sequences were submitted to the GenBank databases (Acc-NO: AY069975 and AY069976; unpublished).

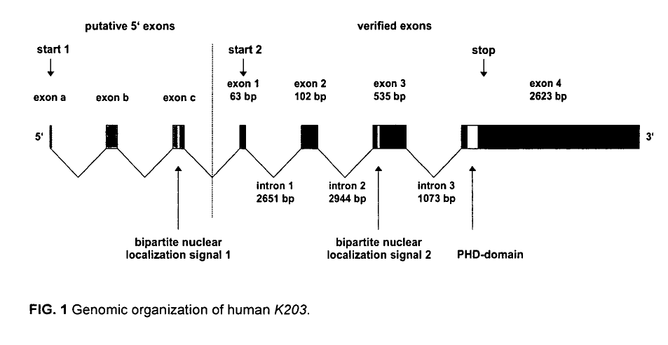

The verified cDNA encodes a putative human protein of 300 amino acids with a predicted molecular weight of approximately 33.5 kDa. The amino acid sequence contains a PHD (plant homeodomain) finger motif preceded by a putative bipartite nuclear localization signal. Interestingly, the three additional exons would be in frame and would add another bipartite nuclear localization signal (FIG. 1). No other significant sequence homologies could be detected by BLAST analysis.

According to the NCBI data K203 is located in chromosome region 1p36.23 (contig NT_{—}028054). Remarkably, chromosome region 1p36 has been implicated in tumorigenesis. Yet unidentified genes in this region play a role in the pathogenesis of ovarian cancer, chondrosarcoma, osteosarcoma, neuroblastoma, endometrial cancer, cervix cancer, germ cell tumors, thyroid cancer, prostate cancer and hematological malignancies. Using gene specific primers and a Mouse/Hamster Radiation Hybrid Panel, murine K203 was located to mouse chromosome 4 at 79 to 81 cM. According to the human-mouse homology map (NCBI) this region is syntenic to human chromosome 1p36.2 indicating that the murine ortholog of K203 was identified. The inventors found out that the K203 protein is involved in the regulation of transcription.

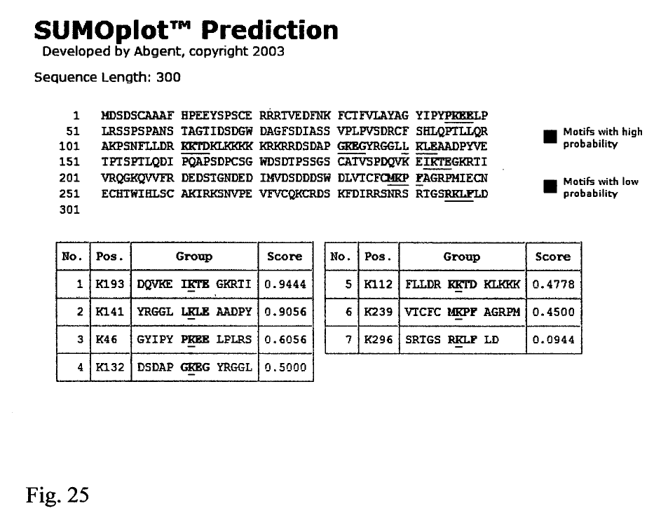

There is a high evidence for the substantial importance of K203:
(i) K203 co-localises with the transcriptional factor E2F1 and with RNA polymerase 11 in the nucleus. This suggests a role of K203 in transcription.
(ii) two new high affinity antibodies against K203 could be generated by the inventors (source: chicken and rabbit), which give the possibility to perform expression studies in the future. In the face of commercial aspects especially these antibodies could be of impact.
(iii) First expression studies showed that K203 is expressed in several different tumor species.
(iv) Comparison of homologies showed that K203 is highly conserved between the species back to Xenopus, which gives evidence for a crucial physiologic function of the factor.
(v) The PHD domain of K203 is a putative SUMo/ubiquitin-ligase domain.
   As this is an enzymatic function, there is a perspective for the identification of inhibitors of this factor. This is of importance for both, the development of a therapeutic strategy and for basic research. K203 itself is regulated by sumolation and has two putative sumolation sites, which probably are important for the activity of K203 (see also FIG. 25).

In particular, the present invention is directed to the following aspects and embodiments:

According to a first aspect, the invention is directed to a human K203 protein, which is encoded by the nucleic acid of SEQ ID NO: 1 or variants thereof, which variants are each defined as having one or more substitutions, insertions, and/or deletions as compared to the nucleic acid of SEQ ID NO: 1, provided that:
a) these variants hybridize under moderately stringent conditions to a nucleic acid, which comprises the sequence of SEQ ID NO: 1, and further provided that these variants code for a protein having K203 activity; or
b) these variants have nucleic acid changes which are due to the degeneration of the genetic code, which code for the same or functional equivalent amino acid as the nucleic acid of SEQ ID NO: 1.

The invention is further directed to the murine K203 protein, which is encoded by the nucleic acid of SEQ ID NO: 2 or variants thereof, wherein the variants are each defined as having one or more substitutions, insertions and/or deletions as compared to the sequence of SEQ ID NO: 2, provided that:
a) said variants hybridize under moderately stringent conditions to a nucleic acid which comprises the sequence of SEQ ID NO: 2, and further provided that said variants code for a protein having K203 activity; or
b) these variants having nucleic acid changes, which are due to the degeneration of the genetic code, which code for the same or a functional equivalent amino acid as the nucleic acid of SEQ ID NO: 2.

Further, the invention provides a human isolated nucleic acid, which comprises the nucleic acid of SEQ ID NO: 1 or variants thereof, wherein the variants are each defined as having one or more substitutions, insertions, and/or deletions as compared to the nucleic acid of SEQ ID NO: 1, provided that:
a) these variants hybridize under moderately stringent conditions to a nucleic acid, which comprises the sequence of SEQ ID NO: 1, and further provided that these variants code for a protein having K203 activity; or
b) said variants have nucleic acid changes which are due to the degeneration of the genetic code, which code for the same or functional equivalent amino acids as the nucleic acid of SEQ ID NO: 1.

The invention is further directed to a murine isolated nucleic acid which comprises the nucleic acid of SEQ ID NO: 2 or variants thereof, wherein the variants are each defined as having one or more substitutions, insertions, and/or deletions as compared to the sequence of SEQ ID NO: 2, provided that:
a) said variants hybridize under moderately stringent conditions to a nucleic acid, which comprises in the sequence of SEQ ID NO: 2, and further provided that these variants code for a protein having K203 activity; or
b) these variants have nucleic acid changes, which are due to the degeneration of the genetic code, which code for the same or a functional equivalent amino acid as compared to the nucleic acid of SEQ ID NO: 2.

The term “K203 activity” or “K203 function” as used herein encompasses all functions, which can be ascribed to this protein in its in vivo context. Generally, this function can be broadly defined as a transcriptional repressor activity.

The transcriptional repressor activity of K203 (synonym: SPOC1) and all above described variants, falling within the scope of this invention, can be tested by constructing a plasmid expressing a fusion protein between K203 or its variant and the GAL4 DNA-binding domain (vector systems are commercially available). The yeast GAL4 Protein is a transcriptional activator that binds to DNA. The construct is co-transfected into 293T cells or other cells with a low endogenous K203 expression together with a luciferase-reporter plasmid containing GAL4 DNA-binding elements in front of a luciferase reporter gene. The GAL4 DNA-binding domain binds to the corresponding DNA-binding elements in the reporter plasmid and activates luciferase expression. Luciferase activity can be measured with a luminometer. The fused K203 protein (or variant thereof) represses this transcription. The degree of repression can be determined by comparison with a control plasmid (GAL4 DNA-binding domain alone).

The nucleic acid variants according to the invention also comprise nucleic acid fragments which contain more than 10, preferably more than 15, more than 20, more than 25 or more than 30 and up to 50 nucleotides. The term oligonucleotide includes fragments containing 10 to 50 nucleotides and parts thereof. These sequences can be in any order as long as at least 10 successive nucleotides are according to the invention. These oligonucleotides can be preferably used as primers, for example for RT-PCR or as a probe for in situ hybridization.

According to the state of the art an expert can test which derivatives and possible variations derived from these revealed nucleic acid sequences according to the invention are, are partially or are not appropriate for specific applications like hybridization and PCR assays. The nucleic acid and oligonucleotides of the inventions can also be part of longer DNA or RNA sequences, e.g. flanked by restriction enzyme sites.

Amplification and detection methods are according to the state of the art. The methods are described in detail in protocol books which are known to the expert. Such books are for example Sambrook et al., 1989, Molecular Cloning, A Laboratory Manual, Cold Spring Harbor Press, and all subsequent editions. PCR-methods are described for example in Newton, PCR, BIOS Scientific Publishers Limited, 1994 and all subsequent editions.

As defined above, “variants” are according to the invention especially such nucleic acids, which contain one or more substitutions, insertions and or deletions when compared to the nucleic acids of SEQ ID NO: 1 and 2. These lack preferably one, but also 2, 3, 4, or more nucleotides 5′ or 3′ or within the nucleic acid sequence, or these nucleotides are replaced by others.

The nucleic acid sequences of the present invention also comprise such nucleic acids which contain sequences in essence equivalent to the nucleic acids described in SEQ ID NO: 1 and 2. According to the invention nucleic acids can show for example at least about 80%, more typically at least about 90% or 95% sequence identity to the nucleic acids described in SEQ ID NO: 1 and 2.

The term “nucleic acid sequence” means a heteropolymer of nucleotides or the sequence of these nucleotides. The term “nucleic acid”, as herein used, comprises RNA as well as DNA including cDNA, genomic DNA and synthetic (e.g. chemically synthesized) and to other polymers linked bases such as PNA (peptide nucleic acids) or PTO (phosphothioat oligonucleotides).

The invention comprises—as mentioned above—also such variants which hybridize to the nucleic acids according to the invention at moderate stringent conditions.

Stringent hybridization and wash conditions are in general the reaction conditions for the formation of duplexes between oligonucleotides and the desired target molecules (perfect hybrids) or that only the desired target can be detected. Stringent washing conditions mean for example 0.2×SSC (0.03 M NaCl, 0.003 M sodium citrate, pH 7)/0.1% SDS at 65° C. For shorter fragments, e.g. oligonucleotides up to 30 nucleotides, the hybridization temperature is below 65° C., for example at 50° C., preferably above 55° C., but below 65° C. Stringent hybridization temperatures are dependent on the size or length, respectively of the nucleic acid and their nucleic acid composition and will be experimentally determined by the skilled artisan. Moderate stringent hybridization temperatures are for example 42° C. and washing conditions with 0.2×SSC/0.1% SDS at 42° C.

The respective temperature conditions can vary dependent on the chosen experimental conditions and to be tested nucleic acid probe, and have to be adapted appropriately. The detection of the hybridization product can be done for example using X-Ray in the case of radioactive labeled probes or by fluorimetry in the case of fluorescent labeled probes.

The expert can according to the state of the art adapt the chosen procedure, to reach actually moderate stringent conditions and to enable a specific detection method. Appropriate stringent conditions can be determined for example on the basis of reference hybridization. An appropriate nucleic acid or oligonucleotide concentration needs to be used. The hybridization has to occur at an appropriate temperature (the higher the temperature the lower the binding).

As mentioned above, fragments of the nucleic acids according to the invention can be used for example as oligonucleotide primer in detection systems and amplification methods of the K203 gene and K203 transcript. The expert can apply these oligonucleotides in state of the art methods. DNA or RNA can be analyzed for the presence of one of the described genes or transcripts applying the appropriate oligonucleotide primers to the to be analyzed probe. The detection of the RNA or DNA of the probe can be achieved for example by PCR methods, which reveal the presence of the specific DNA and/or RNA sequences. All hereinabove described oligonucleotides can also be used as primers, also as primers for reverse transcription of RNA.

The PCR method has the advantage that very small amounts of DNA are detectable. Dependent on the to be analyzed material and the equipment used the temperature conditions and number of cycles of the PCR have to be adjusted. The optimal conditions can be experimentally determined according to standard procedures.

The during the PCR amplification accrued, characteristic, specific DNA fragments can be detected for example by gel electrophoretic or fluorimetric methods with the DNA labeled accordingly. Alternatively, other appropriate, known to the expert, detection systems can be applied.

The DNA or RNA, especially mRNA, of the to be analyzed probe can be an extract or a complex mixture, in which the DNA or RNA to be analyzed are only a very small fraction of the total biological probe. This probe can be analyzed by PCR, e.g. RT-PCR or in hybridization assays. The biological probe can be serum, blood or cells, either isolated or for example as mixture in a tissue. Further, the herein described oligonucleotides can be used for RT-PCR, in situ PCR, in situ RT-PCR or in situ hybridization.

In the case of RT-PCR oligonucleotides of the invention are used for PCR amplification of fragments of cDNA matrices, which resulted from the reverse transcription of probe RNA or mRNA. The expression analysis can be qualitative or together with appropriate controls and methods quantitative. For the quantitative analyses an internal standard is used.

According to an embodiment of the invention, the isolated nucleic acid according to the invention is further operably linked to one or more regulatory sequences.

The present invention comprises further transcriptional products of the hereinabove described nucleic acids and nucleic acids, which selectively hybridize under moderate stringent conditions to one of these transcriptional products. Preferably this comprises siRNA or an antisense DNA or RNA in form of a DNA or RNA probe which can hybridize to a transcription product, e.g. mRNA, and can be used in detection systems.

The term “probe” is here defined as a nucleic acid which can bind to a target nucleic acid via one or more kind of chemical binding, usually via complementary base pairing which usually form hydrogen bonds.

For detection the nucleic acids according to the invention are preferably labeled, for example with radioactive labeling, dyeings, fluorophore labeling, enzyme labeling and the like. Preferred examples for labeling are digoxygenin, biotin, peroxidase, fluorescence or alkaline phosphatase. Depending on the label, the detection can be direct or enhanced using indirect immunohistochemistry. Alkaline phosphatase is used as marker enzyme since it develops a sensitive, striking color reaction in the presence of appropriate substrates. Substrates, like p-nitrophenylphosphate, are cleaved and release colored, photometrically measurable products.

In a further embodiment, the present invention provides nucleic acids coupled to a matrix, e.g. nylon membrane, nitro cellulose membranes, glass or polymers.

From a protein point of view (also having regard of the amino acid sequence of SEQ ID NO:3), the invention encompasses such changes in the nucleic acid sequence which are considered to cause a substitution with a functionally equivalent amino acid (for an assay, see above). Preferably are such amino acid substitutions the result of substitutions which substitute one amino acid with a similar amino acid with similar structural and/or chemical properties, i.e. conservative amino acid substitutions. Any amino acid exchange will be regarded as being equivalent in the meaning of the invention, which leads to a protein having K203 activity.

Amino acid substitutions can be performed on the basis of similarity in polarity, charges, solubility, hydrophobic, hydrophilic, and/or amphipathic (amphiphil) nature of the involved residues. Examples for hydrophobic amino acids are alanine, leucine, isoleucine, valine, proline, phenylalanine, tryptophan and methionine. Polar, neutral amino acids include glycine, serine, threonine, cysteine, thyrosine, asparagine and glutamine. Positively (basic) charged amino acids include arginine, lysine and histidine. And negatively charged amino acids include aspartic acid and glutamic acid.

“Insertions” or “deletions” usually range from one to five amino acids. The allowed degree of variation can be experimentally determined via methodically applied insertions, deletions or substitutions of amino acids in a polypeptide molecule using recombinant DNA methods. The resulting variants can be tested for their biological activity. “Insertions” or “deletions” are typically in the range of about 1 to 5 amino acids.

Nucleotide changes, which affect the N-terminal and C-terminal part of the protein, often do not change the protein activity, because these parts are often not involved in the biological activity. It can be desired to eliminate one or more of the cysteins of the sequence, since cysteines can cause the unwanted formation of multimers when the protein is produced recombinant. Multimers may complicate purification procedures. Each of the suggested modifications is in range of the current state of the art, and under the retention of the biological activity of the encoded products.

In a further embodiment, the present invention includes the invention of a vector (construct) comprising a nucleic acid according to the invention. This vector is preferably an expression vector which contains a nucleic acid according to the invention and one or more regulatory nucleic acid sequences.

Numerous vectors are known to be appropriate for the transformation of bacterial cells, for example plasmids and bacteriophages, like the phage λ, are frequently used as vectors for bacterial hosts. Viral vectors can be used in mammalian and insect cells to express exogenous DNA fragments, e.g. SV 40 and polyoma virus.

The transformation of the host cell can be done alternatively directly using “naked DNA” without the use of a vector.

The protein according to the invention can be produced either in eukaryotic or prokaryotic cells. Examples for eukaryotic cells include mammalian, plant, insect and yeast cells. Appropriate prokaryotic cells include Escherichia coli and Bacillus subtilis.

Preferred mammalian host cells are CHO, COS, HeLa, 293T, HEH or BHK cells or adult or human or non-human embryonic stem cells.

Alternatively, the protein according to the invention can be produced in transgenic plants (e.g. potatoes, tobacco) or in transgenic animals, for example in transgenic goats or sheep.

In a further embodiment, the present invention includes an antibody or aptamer which recognizes K203 protein according to the invention.

The antibody is preferably selected from a group, which consists of polyclonal antibodies, monoclonal antibodies, humanized antibodies, chimeric antibodies and synthetic antibodies.

The antibody according to the invention can be additionally linked to a toxic and/or a detectable agent.

The term “antibody” is used herein for intact antibodies as well as antibody fragments, which have a certain ability to selectively bind to an epitop. Such fragments include, without limitations, Fab, F(ab′)₂ and Fv antibody fragment. The term “epitop” means any antigen determinant of an antigen, to which the paratop of an antibody can bind. Epitop determinants usually consist of chemically active surface groups of molecules (e.g. amino acid or sugar residues) and usually display a three-dimensional structure as well as specific physical properties.

The antibodies according to the invention can be produced according to any known procedure. For example the pure complete protein according to the invention or a part of it can be produced and used as immunogen, to immunize an animal and to produce specific antibodies.

The production of polyclonal antibodies is commonly known. Detailed protocols can be found for example in Green et al, Production of Polyclonal Antisera, in Immunochemical Protocols (Manson, editor), pages 1-5 (Humana Press 1992) and Coligan et al, Production of Polyclonal Antisera in Rabbits, Rats, Mice and Hamsters, in Current Protocols In Immunology, section 2.4.1 (1992). In addition, the expert is familiar with several techniques regarding the purification and concentration of polyclonal antibodies, as well as of monoclonal antibodies (Coligan et al, Unit 9, Current Protocols in Immunology, Wiley Interscience, 1994).

The production of monoclonal antibodies is as well commonly known. Examples include the hybridoma method (Kohler and Milstein, 1975, Nature, 256:495-497, Coligan et al., section 2.5.1-2.6.7; and Harlow et al., Antibodies: A Laboratory Manual, page 726 (Cold Spring Harbor Pub. 1988).), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

In brief, monoclonal antibodies can be attained by injecting a mixture which contains the protein according to the invention into mice. The mice used can be also a transgenic mouse or a mouse deficient in K203. The antibody production in the mice is checked via a serum probe. In the case of a sufficient antibody titer, the mouse is sacrificed and the spleen is removed to isolate B-cells. The B cells are fused with myeloma cells resulting in hybridomas. The hybridomas are cloned and the clones are analyzed. Positive clones which contain a monoclonal antibody against the protein are selected and the antibodies are isolated from the hybridoma cultures. There are many well established techniques to isolate and purify monoclonal antibodies. Such techniques include affinity chromatography with protein A sepharose, size-exclusion chromatography and ion exchange chromatography. Also see for example, Coligan et al., section 2.7.1-2.7.12 and section “Immunglobulin G (IgG)”, in Methods In Molecular Biology, volume 10, pages 79-104 (Humana Press 1992).

According to a still further embodiment, the invention as hereinabove described provides a hybridoma cell line which produces a monoclonal antibody which specifically binds to a K203 protein according to the invention.

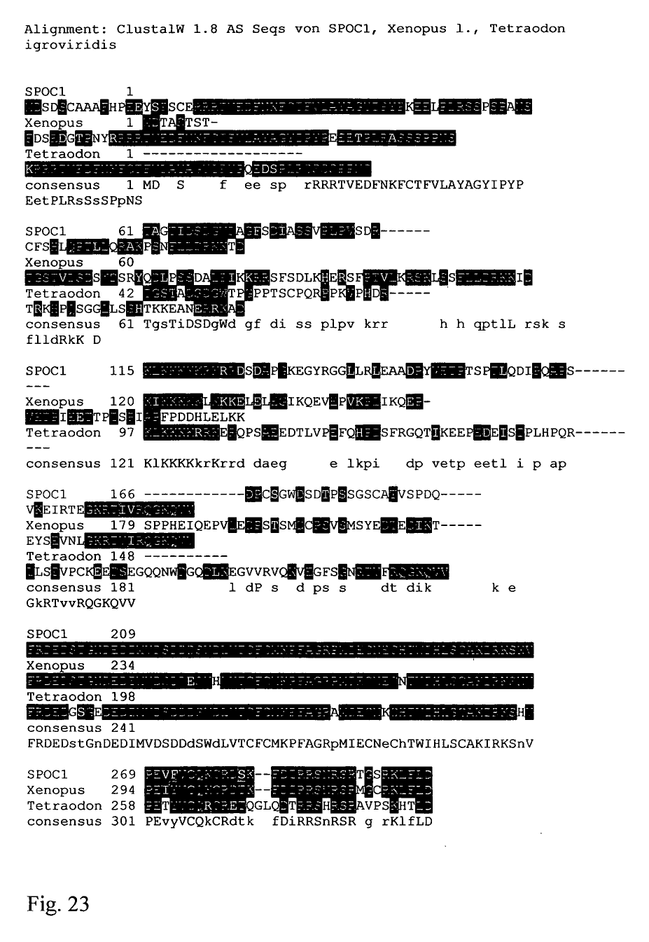

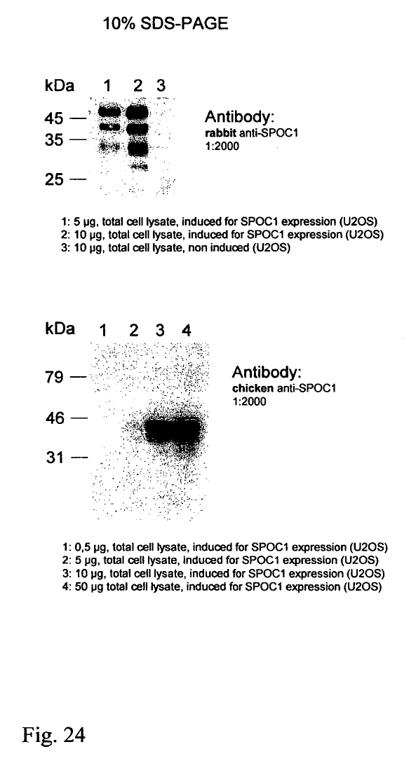

In this invention, an antibody is in particular preferred, which is specific for the amino acid of SEQ ID NO:3 (see FIG. 23) or a part thereof, for example an epitope thereof. In this invention, two new polyclonal antibodies are disclosed, which specifically bind to that sequence (rabbit and chicken, see chapter Example). High affinity antibodies against SPOC1 could be generated in both rabbit and chicken egg yolk (FIG. 24). The availability of SPOC1 specific antibodies will simplify the expression studies for the future.

According to a further aspect, additional K203-binding compounds can be used, in particular small molecules, recombinant phages, or peptides. Suitable molecules are e.g., anticalins, described in EP1017814. Said European patent also describes the process of preparing such anticalins with the ability to bind a specific target. Further suitable molecules are Trinectins (Phylos Inc., Lexington, Mass., USA, and Xu et al., Chem. Biol. 9:933, 2002). Another kind of suitable molecules are affybodies (see Hansson et al., Immunotechnology 4(3-4):237-52, 1999, and Henning et al., Hum Gene Ther. 13(12):1427-39, 2002, and references therein). Furthermore, K203 binding peptides, spiegelmere, aptazymes, and ribozymes may be used.

A further approach for blocking the K203 protein according to the invention is the RNAi technology. As its name suggests, RNA interference (RNAi) is a cellular mechanism to regulate the expression of genes and the replication of viruses. This mechanism is mediated by double-stranded small interfering RNA molecules (siRNA). Messenger RNA (mRNA) provides the means of implementing the set of instructions contained within the genetic material to produce the cell's machinery. Altering the function of the mRNA therefore can be used to modulate the cell's machinery. RNAi technology is a comparatively recent discovery which constitutes an important aspect of a cell's natural defensive mechanism against, e.g. parasitic viruses. Critically, the cell responds to a foreign (double stranded) form of siRNA introduced into the cell by destroying all internal mRNA with the same sequence as the siRNA.

The invention further includes a pharmaceutical composition comprising a nucleic acid according to the invention, a vector, an antibody or aptamer according to the invention as an active component in combination with a pharmaceutical acceptable carrier.

The active components of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” is defined as non-toxic material, which does not interfere with the effectiveness of the biological activity of the active component. The choice of the carrier is dependent on the application.

The pharmaceutical composition can contain additional components which enhance the activity of the active component or which supplement the treatment. Such additional components and/or factors can be part of the pharmaceutical composition to achieve a synergistic effects or to minimize adverse or unwanted effects.

Techniques for the formulation or preparation and application/medication of compounds of the present invention are published in “Remington's Pharmaceutical Sciences”, Mack Publishing Co., Easton, Pa., latest edition. A therapeutically effective dose relates to the amount of a compound which is sufficient to improve the symptoms, for example a treatment, healing, prevention or improvement of such conditions. An appropriate application can include for example oral, dermal, rectal, vaginal, transmucosal or intestinal application and parenteral application, including intramuscular, subcutaneous, intramedular injections as well as intrathecal, direct intraventricular, intravenous, intraperitoneal or intranasal injections. The intravenous injection is the preferred treatment of a patient.

A typical composition for an intravenous infusion can be produced such that it contains 250 ml sterile Ringer solution and for example 10 mg K203 protein. See also Remington's Pharmaceutical Science (15. edition, Mack Publishing Company, Easton, Ps., 1980).

The active component or mixture of it in the present case can be used for prophylactic and/or therapeutic treatments.

An amount which is adequate to reach the aforesaid effect is defined as “therapeutically effective dose”. Amounts, which are effective for these applications, depend on the severity of the condition and the general condition of the patient and his immune system. However, the dose range is usually between 0.01 and 100 mg protein and, preferably between 0.1 to 50 mg and most preferably from 1 to 10 mg per patient. Single or multiple applications after a daily, weekly or monthly treatment regimen can be performed with application rate and samples chosen by the physician in charge.

In a further embodiment, the present invention includes a diagnostic composition which contains an antibody, aptamer or any other K 203 binding compound as indicated above or probe according to the invention.

Further, the invention includes a transgenic, non-human mammal, which has one or more K203 sequences according to the inventions inactivated. Using the homologous recombination technology as described for example in “(Gene Targeting: A Practical Approach” (editor A. Joyner, Oxford University Press, 2nd edition, 2002) or “Gene Knockout Protocols” (editor M. J. Tymms and I. Kola, Humana Press, 1st edition 2001), a knock-out animal model can be established. This will enable to elucidate further functions of K203 and especially the etiology of cancer.

The invention comprises preferably a transgenic mouse with a nucleic acid of the invention conditionally and permanently inactivated. The conditional knock out is a special case within the knock-out technology. The original knock-out technology applications result in the constitutively deletion of the gene to be analyzed. In the present invention a system can be used to create a cell type-specific and/or temporally controlled conditionally inactivation of a gene in a specific tissue or cell type at a specific time point. For the conditional gene inactivation in a certain tissue a specific promoter is necessary to disable the desired gene in the selected tissue or cells. For example a K203 promoter can be used to inactivate selected genes. To achieve this the K203 promoter according to the invention will be ligated at the DNA level to an appropriate recombinase, for example Cre of flp. This construct may further include other regulatory sequences to guarantee the expression of the recombinase. The construct can be tested in vitro before it is used to produce transgenic, non-human animals, preferably transgenic mice. The founder mice will be analyzed for correct expression of the recombinase in the specific tissue or cells, for example ovary, and the positive ones will be later used for intercrossing. Genes to be cell- or tissue-specific inactivated are cloned into vector such that the regions to be deleted are flanked by recombinase recognition site, for example IoxP for the Cre recombinase and frt for the Flp recombinase. Using the knock-out technology the vector is transfected into embryonic stem (ES) cells and clones with the correct integrations are selected and used for the production of chimeric animals. The heterozygous or homozygous offspring of these will be intercrossed with transgenic mice containing the recombinase resulting in a tissue-specific deletion of the selected gene. The effects can be analyzed and will lead to a further understanding of cancer. With the use of a K203 promoter the effect of genes can be analyzed leading to a greater understanding of cancer.

Further, the present invention provides a non-human transgenic mammal, which has a nucleic acid according to the invention inserted. For example can the K203 cDNA be ectopically expressed to investigate activities of K203 in other tissues. Further can the K203 promoter nucleic acid according to the invention be ligated to other cDNAs or genes and other regulatory sequences to overexpress these cDNAs or genes in specific tissues. This method can be applied for target identification and validation to develop potential novel treatments for cancer diseases.

Furthermore, the present invention provides a diagnostic composition, comprising a K203 binding molecule, e.g. an antibody, an aptamer or a small molecule as defined herein. As an alternative, a diagnostic composition is provided comprising a probe of the invention.

Furthermore, the invention is directed to an ex-vivo method for the diagnosis of cancer comprising the following steps:
a) providing a tissue sample or a serum sample from a patient;
b) qualitative and/or quantitative determination of the transcriptional products of the invention or of the K203 protein of the invention in the sample; wherein
an overexpression of the transcriptional products or of the K203 protein in the tissue or serum sample is indicative for the presence of cancer and the degree of expression is indicative for the prognosis of said patient.

The analysis in step b) is preferably done by Northern Blot, in situ hybridization or RT-PCR, in situ RT-PCR preferably semiquantitative RT-PCR, or a combination thereof. For further details see also McPherson et al. (ed.), PCR, A Practical Approach, Oxford, IRL Press 1995.

Further the analysis in step b) can be done using a diagnostic composition as hereinabove described with K203 binding compounds, e.g. anti K203 antibodies, small molecules or aptamers or using specific DNA or RNA probes as defined above for K203 according to the invention.

A main aspect of the invention resides in a method of treating cancer, comprising administering an therapeutically effective amount of the pharmaceutical composition as defined herein to a patient in need of such treatment. For example, an antibody linked to a toxic and/or therapeutic means may be used to direct that antibody to cancer cells, where the toxic and/or therapeutic means can act in killing those cancer cells.

The method is preferably used for the treatment of cancer, wherein the cancer is selected from ovarian cancer, chondrosarcoma, osteosarcoma, neuroblastoma, endometrial cancer, cervix cancer, germ cell tumors, thyroid cancer, lung cancer, prostate cancer, colon cancer, kidney cancer, bladder cancer, esophageal cancer, rectal cancer, meningioma and other tumors of the central nervous system, parathyroid cancer, hepatocellular cancer and hematological malignancies. It is noted that the invention preferably finds application regarding ovarian cancer and colon cancer.

According to a further aspect, a human K203 protein having the amino acid sequence of SEQ ID NO: 3 or a variant of said sequence is provided, wherein said variant comprises one or more insertions, substitutions and/or deletions as compared to the sequence of SEQ ID NO: 3, and wherein the biological activity is substantially equal to the activity of the protein comprising the unmodified amino acid sequence of SEQ ID NO: 3.

As mentioned above, the PHD domain of K203 is a putative SUMo/ubiquitin-ligase domain. As this is an enzymatic function, there exists the possibility for the identification of inhibitors of this factor. This is of importance for both, the development of a therapeutic strategy and for basic research. K203 itself is regulated by sumolation and has two putative sumolation sites, which probably are important for the activity of K203 (see also FIG. 25).

Thus, the present invention is further directed to a screening method for identifying an antagonist capable of inhibiting or blocking the K203 protein as defined herein, comprising the steps of:
(a) generating or providing mammalian, preferably human, K203,
(b) contacting said K203 with a candidate compound,
(c) detecting the inhibition or blocking of said compound by a suitable detection method,
(d) selecting a compound that has been tested positive in step (c),
(e) optionally repeating steps (a)-(d) with a suitably modified form of the compound of step (d).

Inhibitors will be identified by screening of a substance bank. Purified K203 protein as well as cells expressing K203 will be incubated with substances to identify small molecules that inhibit the sumolation activity of K203.

According to a further aspect, the invention provides a compound, which is capable of inhibiting or blocking the K203 protein as defined herein and/or which is obtainable by the method disclosed above.

The present invention will be further described with reference to the following figures and examples; however, it is to be understood that the present invention is not limited to such figures and examples.

FIG. 1 Genomic organization of human K203.

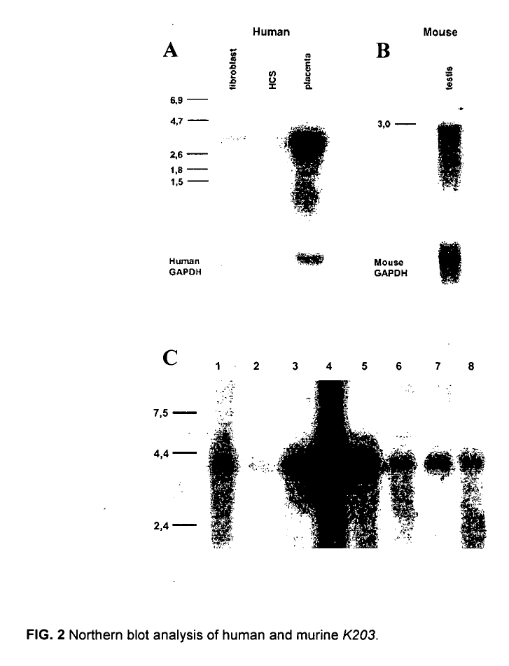

FIG. 2 Northern blot analysis of human and murine K203.

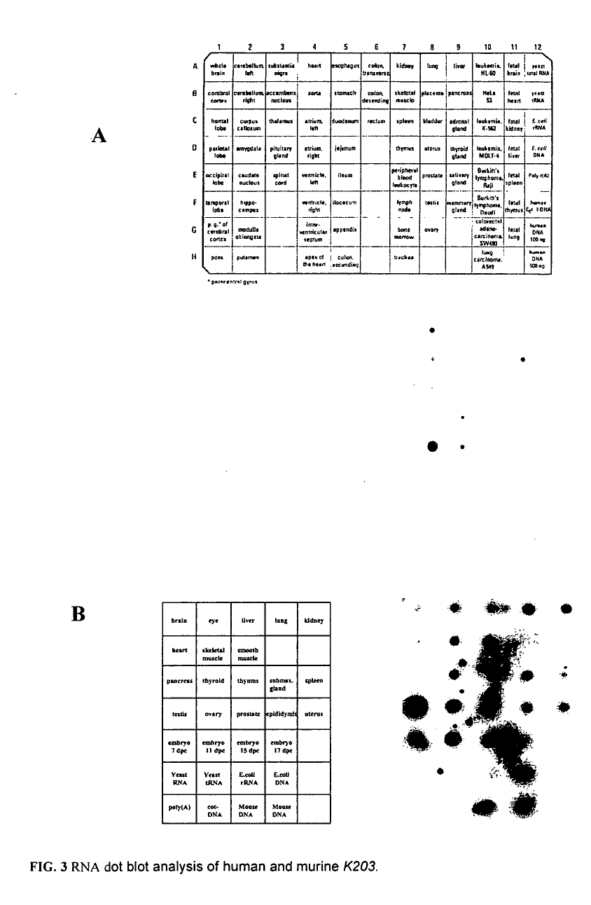

FIG. 3 RNA dot blot analysis of human and murine K203.

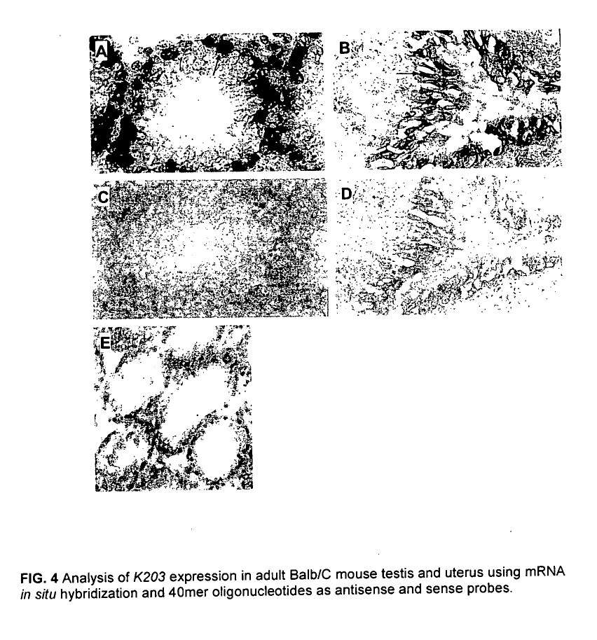

FIG. 4 Analysis of K203 expression in adult Balb/C mouse testis and uterus using mRNA in situ hybridization and 40 mer oligonucleotides as antisense and sense probes.

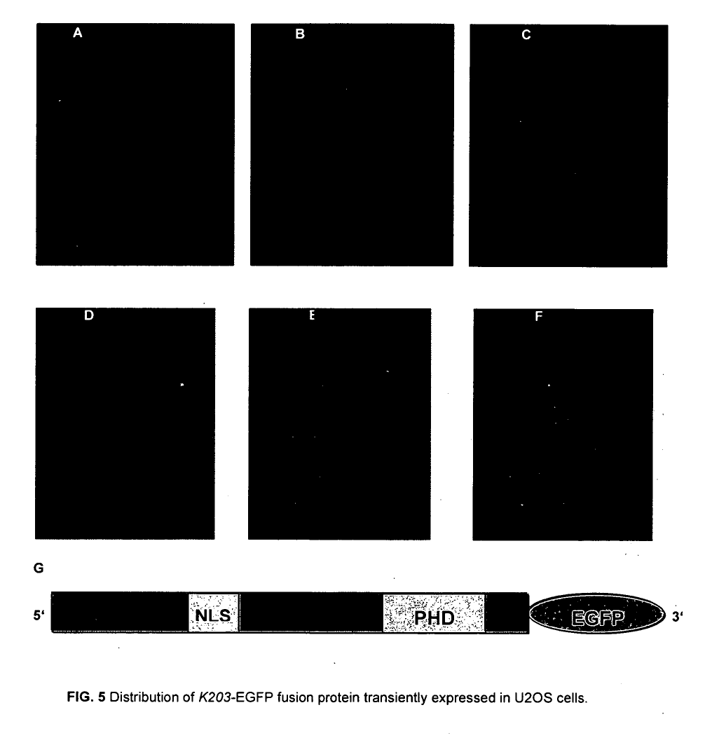

FIG. 5 Distribution of K203-EGFP fusion protein transiently expressed in U2OS cells.

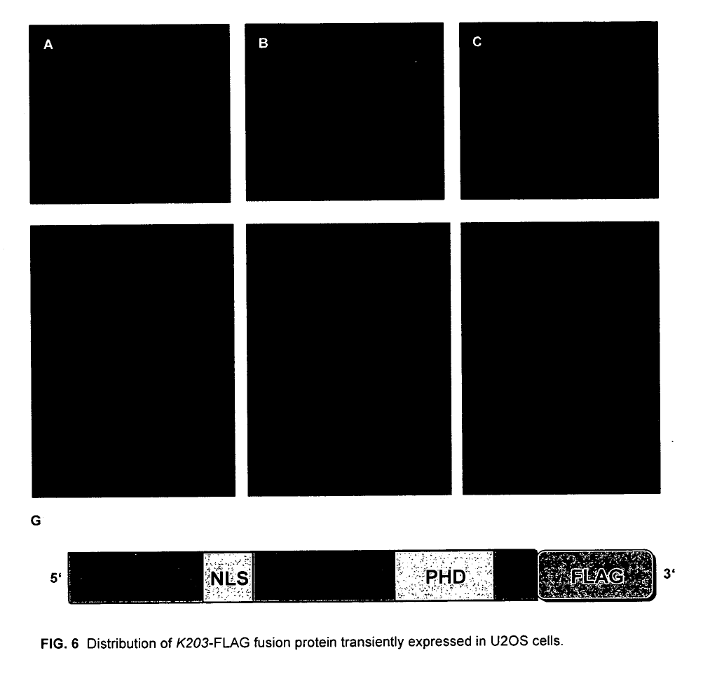

FIG. 6 Distribution of K203-FLAG fusion protein transiently expressed in U2OS cells.

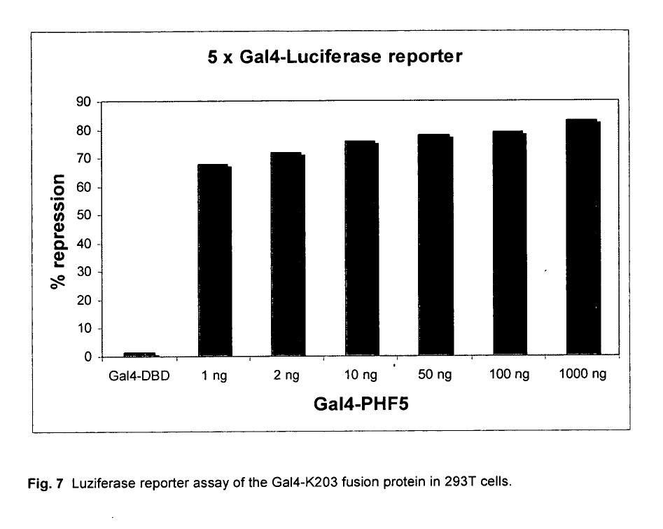

FIG. 7 Luziferase reporter assay of the Gal4-K203 fusion protein in 293T cells.

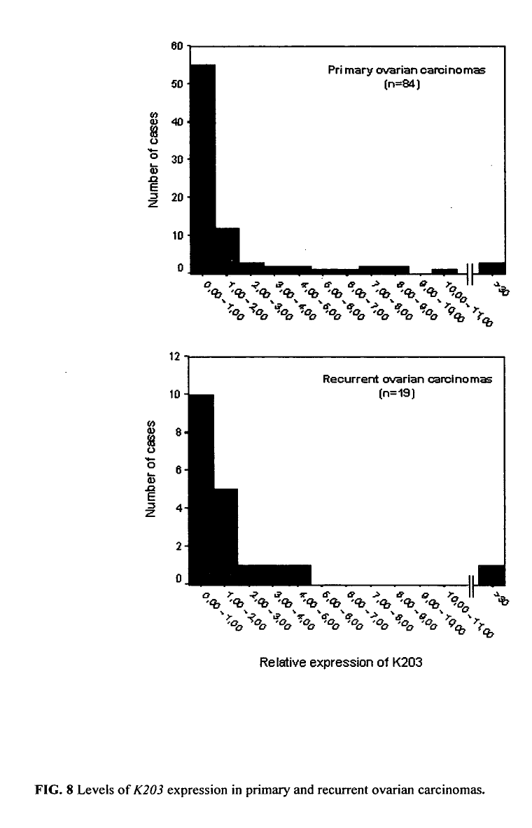

FIG. 8 Levels of K203 expression in primary and recurrent ovarian carcinomas.

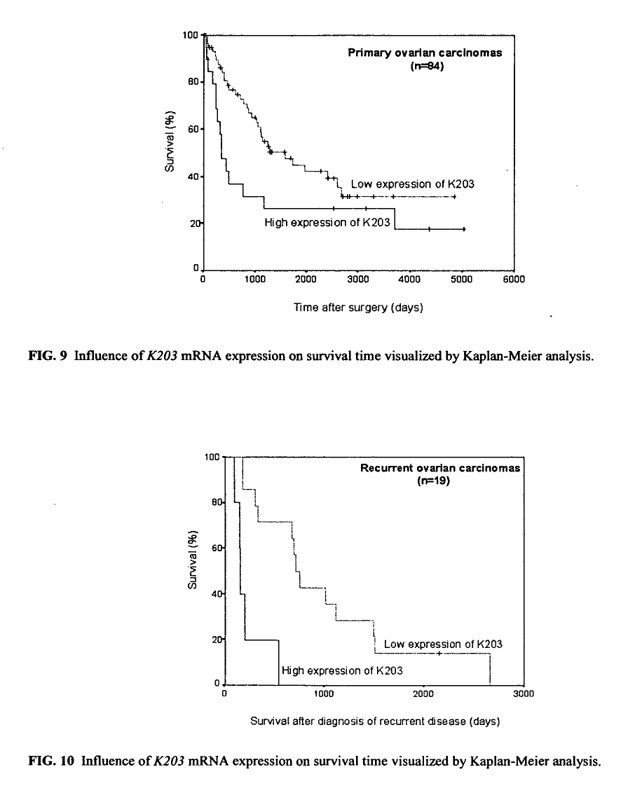

FIG. 9 Influence of K203 mRNA expression on survival time of 84 patients with primary ovarian carcinomas visualized by Kaplan-Meier analysis

FIG. 10 Influence of K203 mRNA expression on survival time of 19 patients with recurrent ovarian carcinomas visualized by Kaplan-Meier analysis.

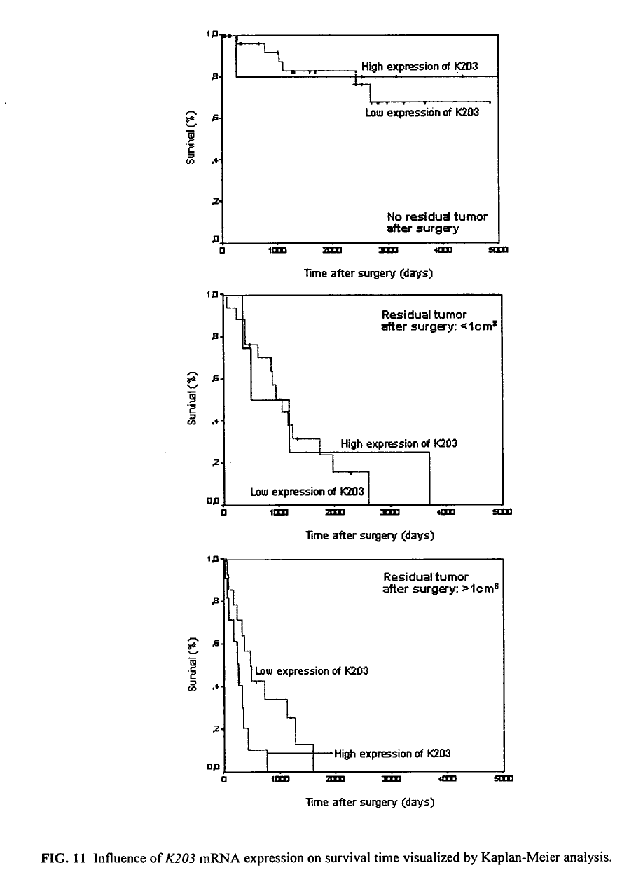

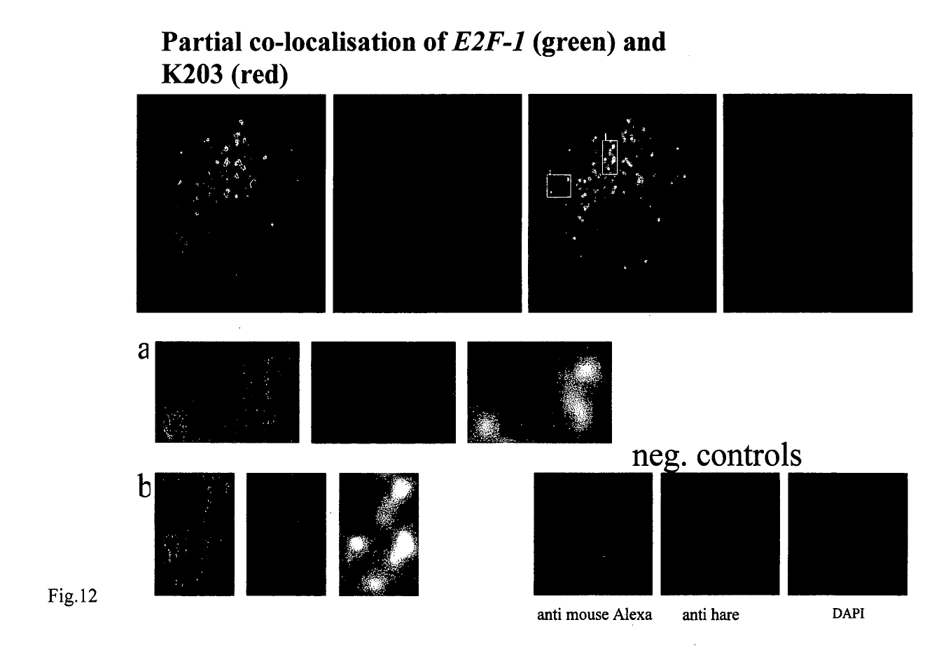

FIG. 11 Influence of K203 mRNA expression on survival time of 84 patients with primary ovarian carcinomas in relation to the residual tumor volume after surgery.

FIG. 12: partial co-localization of E2F-1 (green) and K203 (red) Deuser PT-cell line.

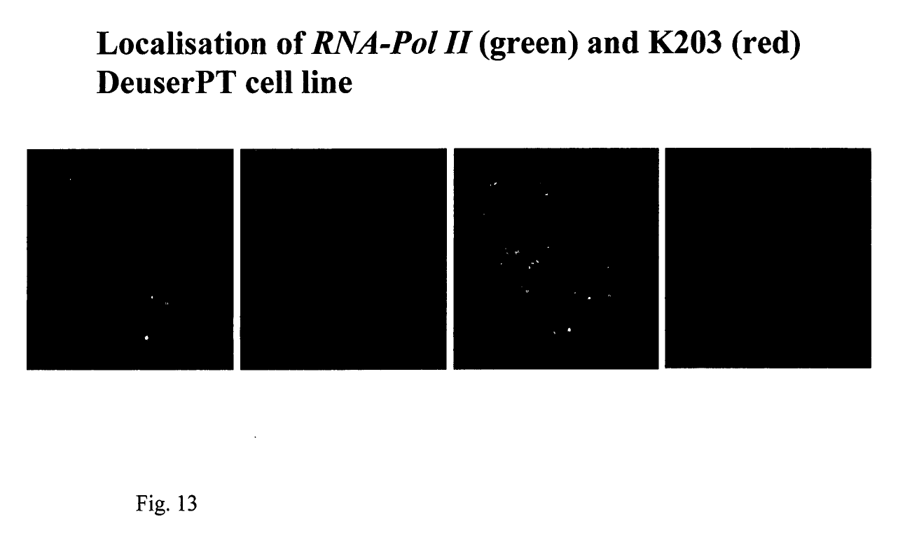

FIG. 13: localization of RNA-Pol II (green) and K203 (red) Deuser PT cell line.

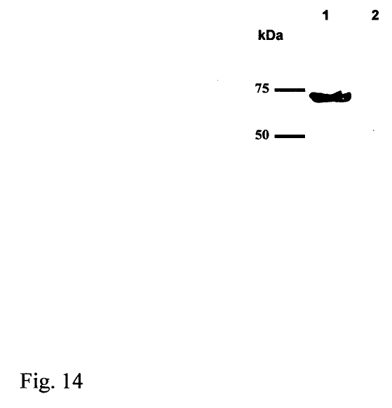

FIG. 14: Western blot analysis of the expression of the SPOC1-EGFP fusion protein in U2OS cells (1). The calculated molecular weight of the fusion protein is 61.2 kDa. Negative control with cell lysate of untransfected U2OS cells (2).

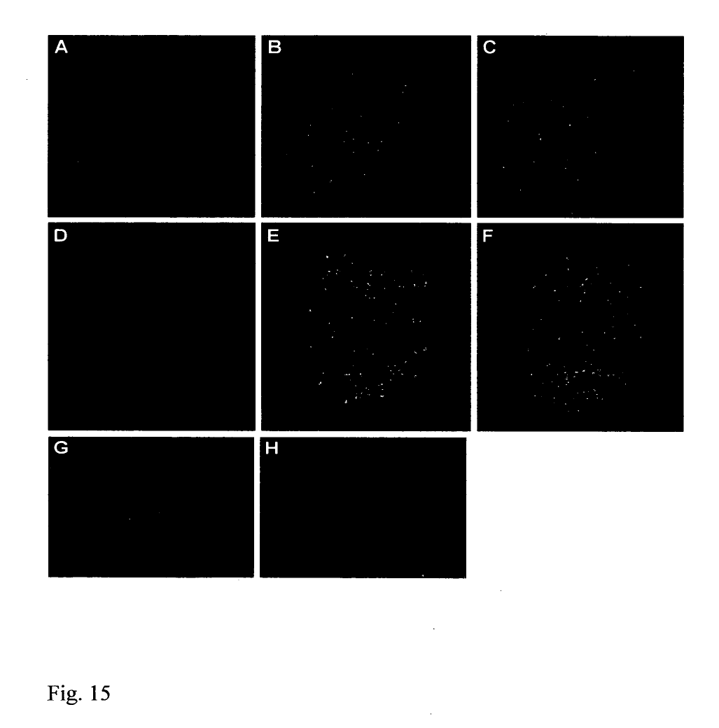

FIG. 15: Subcellular localisation of SPOC1-EGFP (A-C) and FLAG-SPOC1 fusion protein (D-F) in U2OS cells. Both fusion proteins are localised exclusively in the nucleus in form of small speckles. DAPI-stains of the nucleus (A, D), SPOC1-EGFP (B), SPOC1-EGFP and DAPI (C), FLAG-SPOC1 (E), FLAG-SPOC1 and DAPI (F). Transient transfections with the vectors pEGFP-N1 and pFLAG-CMV-4 show a consistently distributed expression of EGFP all over the cell (G) and no expression of the FLAG construct (H).

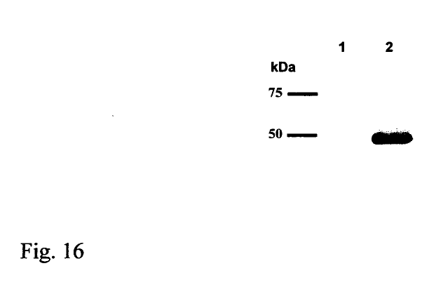

FIG. 16: Expression of the FLAG-SPOC1 fusion protein in U2OS cells. The Western blot analysis of the FLAG-SPOC1 fusion protein shows a slightly aberrant migration behaviour for the fusion protein which can be caused by modifications of the protein (2). The calculated molecular weight of the FLAG-SPOC1 fusion protein is 36.6 kDa. Negative control done with the cell lysate of untransfected U2OS cells (1).

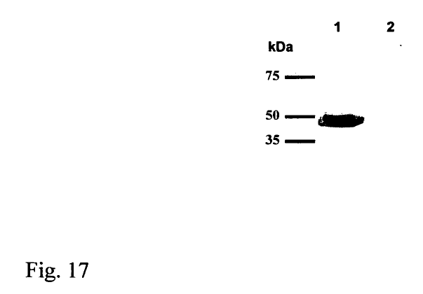

FIG. 17: Detection of the FLAG-SPOC1 fusion protein by the polyclonal SPOC1-#10-Peptide antibody in U2OS-cells (1). Negative control with cell lysate of untransfected U2OS cells (2). The calculated molecular weight of the FLAG-SPOC1 fusion protein is 36.6 kDa. The Western blot analysis of the FLAG-SPOC1 fusion protein shows a slightly aberrant migration behaviour of the fusion protein, which can be caused by the modifications of the protein.

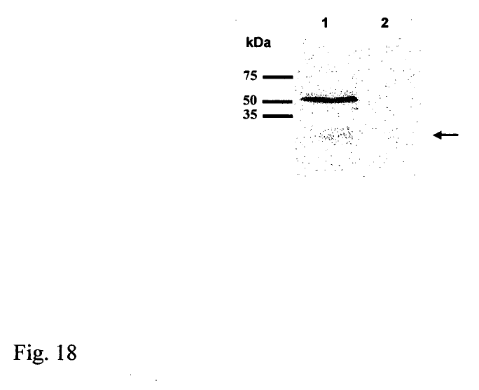

FIG. 18: Detection of the pM2-SPOC1 fusion protein by the polyclonal SPOC1-#10 peptide antibody in U2OS cells (1). The calculated molecular weight of the fusion protein is 51.1 kDa. Negative control with cell lysate of untransfected U2OS cells (2). Unspecific bands in slot 1 and 2 are marked by an arrow.

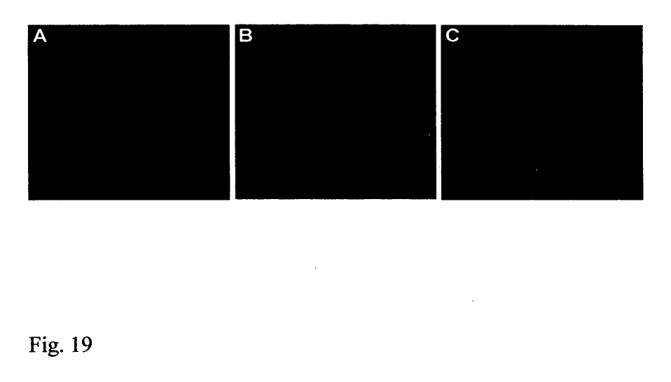

FIG. 19: Subcellular localisation of endogenously expressed SPOC1 protein. SPOC1 is present in the nucleus of untransfected U2OS cells in the form of small speckles distributed all over the nucleus. DAPI (A), SPOC1-#10 (B), SPOC1-#10 und DAPI (C).

Abb. 20: Specifity of the polyclonal SPOC1-#10 peptide antibody. The FLAG-SPOC1 fusion protein is detected in transient transfected cells with both the anti-FLAG antibody and the polyclonal SPOC1-#10 peptide antibody in U2OS cells (A-D) and DeuserPT cells (E-H). DAPI (A, E, I), FLAG-SPOC1 (B, F), SPOC1-#10-peptide antibody (C, G). The overlay of the figures with the anti-FLAG antibody and the polyclonal SPOC1-#10 peptide antibody shows the co-localisation of the signals (yellow) in U2OS cells (D) and DeuserPT cells (H). Transient transfections with the pFLAG-CMV4 construct show no signal (I, J). Negative controls with the SPOC1 antibody show also no signal (data not shown).

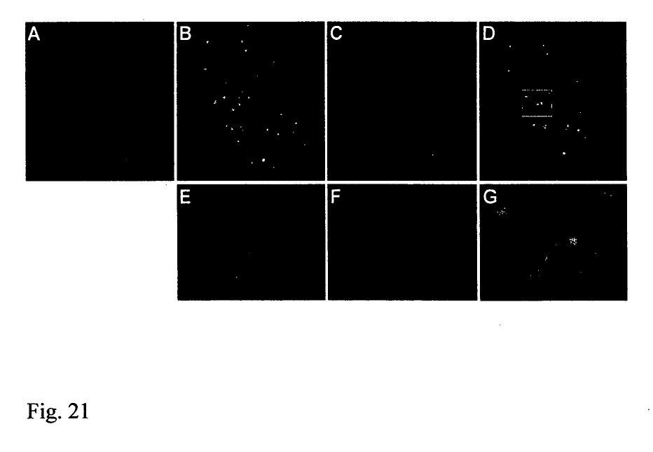

FIG. 21: Clear partial colocalisation of RNA polymerase II and SPOC1 in the nucleus of DeuserPT cells. The comparison of the expression patterns show concordance of the spatial distribution of SPOC1 and RNA polymerase II. DAPI (A), RNA-Polymerase II (B, E) and SPOC1 (C, F). The overlay of the pictures with anti RNA polymerase II antibody and the polyclonal SPOC1 peptide antibody show the partial colocalisation of the signals in yellow (D). The area marked by a rectangle (D) is shown in a 4-fold amplification for RNA polymerase II (E), SPOC1 (F) and for the overlay of both pictures (G).

Abb. 22: Clear partial colocalisation of transcription factor E2F-1 and SPOC1 in the nucleus of DeuserPT cells. DAPI (A), E2F-1 (B), and SPOC1 (C). The overlay of the pictures with the anti-E2F-1 antibody and the polyclonal SPOC1 peptide antibody shows the significant partial colocalisation of the signals in yellow (D). The rectangle (D) marks a 4-fold amplification of a section for E2F-1 (green), SPOC1 (red) and the overlay of the two pictures (yellow). Examples of further colocalisations are marked with arrows (D).

FIG. 23: Comparison of the amino acid sequences of human SPOC1 with the corresponding sequences of Xenopus and Tetraodon, as well as the deduction of a consensus sequence. SPOC1 is highly conserved between the three species.

FIG. 24: Generation and verification of antibodies against SPOC1 raised in rabbit and chicken egg yolk. Western blot analyses of ectopically expressed SPOC1 protein showed that both antibodies detect SPOC1 signals at 36, 40 and 46 kDa in total cell lysates of U2OS-TRex cells.

FIG. 25. Prediction of putative sumoylation target sites in the amino acid sequence of SPOC1 by the SUMOplot prediction software. The two motifs with high probability at positions (LKLE and IKTE) are indicated in red.

### EXAMPLES

Expression Analysis

Northern blot analysis with total RNA from a human chondrosarcoma cell bline, fibroblasts and placenta as well as human multiple tissue northern blot (Clontech) revealed a transcript size of 3.7 kb (FIG. 2A,C). K203 is expressed in all tissues analyzed, with the strongest expression in testis and in placenta. In mouse the transcript size is 3 kb (FIG. 2B). The expression of K203 was also demonstrated in human cartilage samples by RT-PCR. We also performed RNA dot blot analysis (human and mouse multiple tissue expression arrays) with human and mouse K203 as hybridization probes (FIG. 3). Corresponding to the Northern blot hybridizations strongest expression could be detected in human testis and placenta. In mouse the strongest expression could be detected in testis and ovary as well as in embryonal stages 7 dpc and 11 dpc (FIG. 3). Weak expression was detectable in all other tissues. So far, we performed RNA-in situ-hybridization of cryosections from mouse testis and uterus. The experiments revealed a specific expression in spermatogonia and in cells from the functional layer of the endometrium (FIG. 4). Interestingly, both cell types are involved in tumor development (germ cell and endometrial tumors). RNA-in situ-hybridization of mouse ovary sections is currently in progress. So far, RNA-in situ-hybrization of growth plate sections gave no cell type-specific hybridization signals.

Subcellular Localization and GAL4 Assay

We generated K203-EGFP and K203-FLAG fusion constructs and examined the subcellular localization of the fusion proteins by transient transfection in COS-7 and U2OS cells. The fusion proteins are localized solely in the nucleus (FIGS. 5, 6) with a speckled, granular distribution indicating that K203 may be located in specific nuclear domains and may probably be involved in transcriptional regulation.

Using a Gal4-Luciferase reporter assay in 293T cells we could show that increasing amounts of the Gal4-K203 fusion protein resulted in increasing repression of the Gal4-Luciferase reporter gene (FIG. 7) which indicates that K203 functions as a strong transcriptional repressor.

Expression of K203 in Ovarian Cancer

Expression of K203 mRNA was measured in tumor tissue of 84 patients with primary and 19 patients with recurrent ovarian cancer by semiquantitative RT-PCR (Taqman-analysis). Similar levels of K203 mRNA expression were observed in primary and recurrent ovarian carcinomas (FIG. 8). Median expression was 0.65 (0.19-1.45) (25-75% percentiles) in primary carcinomas compared to 0.84 (0.38-1.71) in recurrent tumors. The difference between primary and recurrent ovarian carcinomas was not significant (p=0.125; Wilcoxon-test for unpaired data; two-sided). In addition expression of K203 mRNA was not associated with FIGO-stage, histological grade and type, residual tumor volume after surgery and the age of the patients.

Expression of K203 mRNA was associated with survival of patients with primary ovarian cancer using the univariate proportional hazards model (p=0.001; Table 1A). Relative risk was 1.066 which means that an increase in K203 mRNA expression in tumor tissue by one unit is associated with a 1.026-fold risk to die. As expected the “classical” clinical prognostic factors, namely FIGO-stage, grading and residual tumor volume after surgery were clearly associated with survival (Table 1A). In previous studies with larger case numbers we observed also a weak association between histological type and survival with serous type being associated with worse prognosis. However, no clear influence of histological type was observed for the population examined in the present study.

To examine, whether the influence of K203 is independent from the classical prognostic parameters a multivariable proportional hazards model adjusted for FIGO-stage, histological grade and type as well as residual tumor was performed (Table 1B). Multivariable analysis also showed an association between K203 expression and survival (p=0.064; Table 1B), although this association was weaker compared to that obtained by univariate analysis. However, it should be considered that in the multivariable proportional hazards model the influence of K203 (p=0.064) was stronger compared to that of FIGO-stage and grading (p=0.231 and 0.664, respectively). Similar to several previous studies the residual tumor tissue that could not be removed by surgery was the strongest factor of influence in multivariable analysis (p<0.001; Table 1B).

Since patients with recurrent ovarian cancer do not undergo surgery routinely, the number of tissue specimens available is much lower. However, despite of the low case number (n=19) analyzed in this study, a similar influence of K203 expression on survival of patients with recurrent ovarian carcinomas was obtained compared to primary tumors (p=0.033; Table 2).

The influence of K203 mRNA expression on survival time was visualized by Kaplan-Meier analysis (FIG. 9-11). As in previous studies the 75%-percentile of the factor of interest (K203 mRNA expression) was used as a cutpoint. Patients with primary ovarian cancer with low K203 expression (n=63) survived longer than patients with high K203 expression (n=21) (FIG. 9; p=0.052). Median survival times were 1596 and 347 days for patients with low and high K203 expression, respectively. Although survival times are much shorter for patients with recurrent ovarian cancer a similar influence of K203 was observed (FIG. 10; p=0.0004).

Since multivariable analysis resulted in residual tumor volume as the strongest factor of influence, we performed Kaplan-Meier analysis for subgroups of patients with similar residual disease (FIG. 11). No influence of K203 was seen in patients with no (FIG. 11A) or low (FIG. 11B) residual tumor volume after surgery. Interestingly, the influence of K203 was obvious in patients with more than 2 cm³ tumor left after surgery (FIG. 11C). This type of analysis should be interpreted with caution, since formation of subgroups of patients results in very low numbers of events, especially for patients with no or low residual tumor volume. However, the number of events (n=22 for 27 cases) in the subgroup of patients with more than 2 cm³ tumor left after surgery (FIG. 11C) is relatively high. Due to its homogeneity concerning the strongest prognostic factor “residual disease” and its relatively high number of events this subgroup should be more adequate to analyze the influence of K203 than the subgroups of patients shown in FIGS. 4A and B.

### CONCLUSIONS

K203 is involved in transcriptional regulation, cell cycle control and/or apoptosis (DNA repair) and functions as a transcriptional repressor. We could show that high K203 mRNA expression is associated with worse prognosis for patients with primary and recurrent ovarian cancer, indicating that K203 is involved in the pathogenesis of ovarian or colon cancer. This is strengthened by the fact that K203 is located in a chromosome region implicated in tumorigenesis (including ovarian cancer). Based on the chromosomal localization and/or expression pattern K203 may be involved in the pathogenesis of other tumors as well, like chondrosarcoma, osteosarcoma, neuroblastoma, endometrial cancer, cervix cancer, germ cell tumors, thyroid cancer, lung cancer, prostate cancer, kidney cancer, bladder cancer, esophageal cancer, rectal cancer, meningioma and other tumors of the central nervous system, parathyroid cancer, hepatocellular cancer and hematological malignancies.

Thus, K203 may be used in the aforementioned cancers:
as a marker for disease prognosis and therapy resistance.
for molecular genetic tests aimed at documenting the presence of residual disease after therapy.
as a possible drug target for therapeutic intervention.

Moreover, K203—as a transcriptional regulator—may play a role in numerous other diseases as well as in organogenesis, stem cell growth, virus infection, bacterial infection.

TABLE 1
Association of K203 expression with survival in 84 patients
with primary ovarian cancer using the proportional hazards model
(Cox-analysis)

| | | | |
|---|---|---|---|
| | | 95%-Confidence | |
| Factor | Relative risk | interval | p-value |

A. Univariate analysis

| | | | |
|---|---|---|---|
| K203 mRNA | 1.066 | 1.026-1.107 | 0.001 |
| FIGO-stage (stage III and | 3.322 | 2.821-3.912 | <0.001 |
| IV versus I and II) | | | |
| Grading (grade III vs. | 2.513 | 2.103-3.002 | <0.001 |
| grade II vs. grade II) | | | |
| Histological type (serous vs | 0.858 | 0.681-1.080 | 0.191 |
| non-serous type) | | | |
| Residual tumor after surgery | 3.315 | 2.872-3.826 | <0.001 |
| (0 vs. ≦2 cm³ vs >2 cm³) | | | |

B. Multivariable analysis

| | | | |
|---|---|---|---|
| K203 mRNA | 1.040 | 1.000-1.083 | 0.064 |
| FIGO-stage (stage III and | 1.898 | 0.666-5.415 | 0.231 |
| IV versus I and II) | | | |
| Grading (grade III vs. | 1.121 | 0.670-1.875 | 0.664 |
| grade II vs. grade II) | | | |
| Histological type (serous vs | 1.518 | 0.761-3.027 | 0.236 |
| non-serous type) | | | |
| Residual tumor after surgery | 2.954 | 1.773-4.923 | <0.001 |
| (0 vs. ≦2 cm³ vs >2 cm³) | | | |

TABLE 2
Association of K203 expression with survival in 19 patients with
recurrent ovarian cancer using the proportional hazards model
(Cox-analysis)
Univariate analysis

| | | 95%-Confidence | |
|---|---|---|---|
| Factor | Relative risk | interval | p-value |
| K203 mRNA | 1.912 | 1.055-3.465 | 0.033 |

The present invention is further illustrated by the following experiments conducted by the inventors. It is noted that the protein according to the present invention (K203) is synonymously called SPOC1 in the following:

1. Subcellular Localisation of the SPOC1 Protein

The analysis of the intracellular localisation of the human SPOC1 protein was done with the help of SPOC1-EGFP (enhanced green fluorescent protein) and FLAG-SPOC1 fusion constructs. The SPOC1 reading frame was amplified in PCR experiments with the primers K203 E fw BamHI and K203 E rev BamHI, which contain a BamHI restriction site each. The PCR products were digested with BamHI and the purified product was cloned in the BamHI site of the vectors pEGFP-N1 and pFLAG-CMV-4. The investigation of the subcellular localisation of the fusion proteins was done by transient transfection in COS7-U2OS-, DeuserPT- and BG1p2 cells and fluorescence microscopy 12-24 hours after transfection. Expression profiling in several cell lines (TaqMan analyses) showed that SPOC1 is strongly expressed especially in the oestrogen-receptor positive human ovary carcinoma cell line BG1p2 and in a cell line derived from a human squamous epithelium carcinoma (DeuserPT).

Negative controls were done with transient transfection of the vectors pEGFP-N1 and pFLAG-CMV-4 under the same conditions.

1.1 Subcellular Localisation of the SPOC1-EGFP Fusion Protein

The expression of the SPOC1-EGFP fusion protein with the calculated molecular weight of 61.2 kDa could be confirmed in all cell lines by Western blot analyses and the Living colors A.v-antibody (Clontech, Heidelberg) (results not shown for COS-7, DeuserPT and BG1p2). The results of the Western blot analyses are exemplary shown for U2OS cells (FIG. 14). The analysis of the expression of the SPOC1-EGFP fusion construct in COS-7-, U2OS-, DeuserPT- und BG1p2 cells have shown that the fusion protein is localised almost exclusively in the nucleus in form of small speckles of different size and quantity distributed all over the nucleus (FIG. 15). The localisation of the fusion protein gives evidence for the occurence of SPOC1 in chromatin associated subnuclear domains. The expression pattern of SPOC1-EGFP is identical in all analysed cell lines, therefore only the results for the transient transfections in U2OS cells are shown. The mock transfections with the pEGFP-N1 vector show a consistent distribution of the EGFP protein all over the cell (FIG. 15G).

FIG. 14 shows a Western blot analysis of the expression of the SPOC1-EGFP fusion protein in U2OS cells (1).

FIG. 15: Subcellular localisation of SPOC1-EGFP (A-C) and FLAG-SPOC1 fusion protein (D-F) in U2OS cells. Both fusion proteins are localised exclusively in the nucleus in form of small speckles. DAPI-stains of the nucleus (A, D), SPOC1-EGFP (B), SPOC1-EGFP and DAPI (C), FLAG-SPOC1 (E), FLAG-SPOC1 and DAPI (F). Transient transfections with the vectors pEGFP-N1 and pFLAG-CMV-4 show a consistently distributed expression of EGFP all over the cell (G) and no expression of the FLAG construct (H).

1.3 Subcellular Localisation of the FLAG-SPOC1 Fusion Protein

The expression of the FLAG-SPOC1 fusion protein with the calculated molecular weight of 36.6 kDa could be verified in all cell lines by Western blot analysis and is shown exemplary for U2OS cells in FIG. 16.

The analysis of the localisation of the FLAG-SPOC1 fusion protein in COS-7-, U2OS-, DeuserPT- and BG1p2 cells shows a granulary speckled appearance distributed over the whole nucleus (FIG. 15). To prove the localisation of the FLAG-SPOC1 fusion protein in the nucleus, the nuclei were stained with DAPI. The localisation of the FLAG-SPOC1 fusion protein in the nucleus confirmes therefore the appearance and the localisation of SPOC1-EGFP fusion protein in the analysed cell lines. Further, the localisation of the fusion protein gives evidence for an association with the DAPI stained areas of euchromatin. Control transfection with the pFLAG-CMV-4™ vector showed no specific localisation (FIG. 15 H).

FIG. 16 shows the expression of the FLAG-SPOC1 fusion protein in U2OS cells. The Western blot analysis of the FLAG-SPOC1 fusion protein shows a slightly aberrant migration behaviour for the fusion protein which can be caused by modifications of the protein (2). The calculated molecular weight of the FLAG-SPOC1 fusion protein is 36.6 kDa. Negative control done with the cell lysate of untransfected U2OS cells (1).

1.4 Specificity of the SPOC1 #10 Peptide Antibody

To show the existence of endogenously expressed SPOC1 protein a polyclonal peptide antibody against a peptide sequence of the SPOC1 protein was established. The specificity of the antibody was verified by Western blot analysis with pre-immune serum from rabbit (data not shown) and cell lysates of U2OS- and 293T cells transfected transiently with FLAG-SPOC1- and pM2-SPOC1 constructs. Similarly, untransfected cell lysates of U2OS-, 293T-, DeuserPT-, und BG1p2 cells were analysed in Western blot analyses. Semiquantitative RT-PCR analyses of the expression of SPOC1-mRNA had shown that SPOC1 is overexpressed in the DeuserPT- and BG1p2-cell lines in comparison to the other tested tumour cell lines (MCF7, HeLa, EFO2, EFO21, Lu1I, HIH3T3, NIH3T3-HER2).

Western blot analyses have shown that the SPOC1 peptide antibody is able to detect overexpressed SPOC1 protein in transiently transfected U2OS- and 293T-cells (data not shown for 293T-cells) (FIGS. 4 and 5). The investigation of not transfected cells showed that endogenously expressed SPOC1 is not detected by the SPOC1 peptide antibody (FIGS. 17 and 18, slot 2).

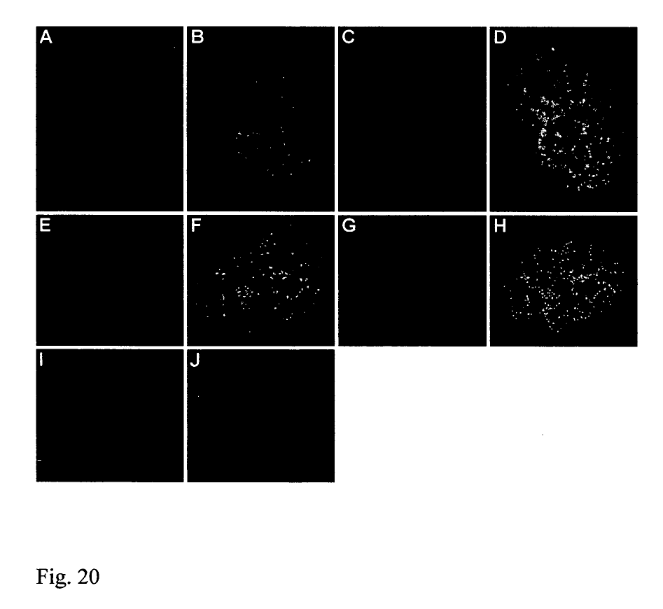

In contrast the analysis of the immunofluorescent experiments has shown that the SPOC1 peptide antibody is able to detect both, endogenously expressed and overexpressed SPOC1 in U2OS-, DeuserPT- and BG1p2 cells. Representative results are shown for U2OS- and DeuserPT cells. The expression pattern detected by SPOC1-#10 in the nucleus is in accordance with the results obtained by the EGFP- and FLAG-SPOC1 constructs (FIG. 19). SPOC1 is present in the nucleus in form of small speckles distributed all over the nucleus. To verify the specificity of the SPOC1 antibody colocalisation experiments were done with U2OS-, DeuserPT- and BG1p2 cells transiently transfected with the FLAG-SPOC1 construct (results not shown for BG1p2) (FIG. 7). Both signals showed a colocalisation (FIG. 20 D, H). The examination of the immunofluorescence showed that the polyclonal SPOC1-#10 peptide antibody detects the same protein as the anti-FLAG antibody.

FIG. 17 shows the detection of the FLAG-SPOC1 fusion protein by the polyclonal SPOC1-#10-Peptide antibody in U2OS-cells (1). Negative control with cell lysate of untransfected U2OS cells (2). The calculated molecular weight of the FLAG-SPOC1 fusion protein is 36.6 kDa. The Western blot analysis of the FLAG-SPOC1 fusion protein shows a slightly aberrant migration behaviour of the fusion protein, which can be caused by the modifications of the protein.

FIG. 18 depicts the detection of the pM2-SPOC1 fusion protein by the polyclonal SPOC1-#10 peptide antibody in U2OS cells (1). The calculated molecular weight of the fusion protein is 51.1 kDa. Negative control with cell lysate of untransfected U2OS cells (2). Unspecific bands in slot 1 and 2 are marked by an arrow.

FIG. 19: Subcellular localisation of endogenously expressed SPOC1 protein. SPOC1 is present in the nucleus of untransfected U2OS cells in the form of small speckles distributed all over the nucleus. DAPI (A), SPOC1-#10 (B), SPOC1-#10 und DAPI (C).

Abb. 20: Specifity of the polyclonal SPOC1-#10 peptide antibody. The FLAG-SPOC1 fusion protein is detected in transient transfected cells with both the anti-FLAG antibody and the polyclonal SPOC1-#10 peptide antibody in U2OS cells (A-D) and DeuserPT cells (E-H). DAPI (A, E, I), FLAG-SPOC1 (B, F), SPOC1-#10-peptide antibody (C, G). The overlay of the figures with the anti-FLAG antibody and the polyclonal SPOC1-#10 peptide antibody shows the co-localisation of the signals (yellow) in U2OS cells (D) and DeuserPT cells (H). Transient transfections with the pFLAG-CMV4 construct show no signal (I, J). Negative controls with the SPOC1 antibody show also no signal (data not shown).

1.5 Colocalisation of SPOC1 with RNA Polymerase II and E2F-1

The distribution of SPOC1 in the nucleus shows a high degree of similarity to the subnuclear domains of different proteins which are associated with transcription. As for PHD-finger proteins a function in the regulation of the chromatin structure is postulated, immunofluorescence experiments were done with the polyclonal SPOC1-#10 peptide antibody and monoclonal antibodies against RNA-polymerase II and the transcription factor E2F-1 in U2OS and DeuserPT cells (data not shown for U2OS cells). The investigation of the immunofluorescences showns a partial but clear colocalization of SPOC1 and RNA polymerase II (FIG. 21). A comparison of the spatial distribution gives evidence that SPOC1 and RNA polymerase II are present in the same subnuclear compartment (FIG. 21).

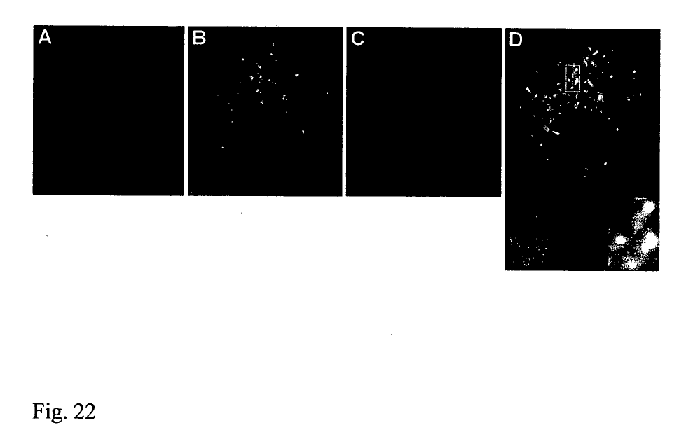

Immunofluorescence experiments with the transcription factor E2F-1 have shown also a partial but clear colocalisation with SPOC1 and confirms at least a partial association with transcriptionally active areas (FIG. 22). The results of the colocalisation experiments thus show a clear association of SPOC1 with subnuclear domains which are associated with the regulation of the transcription.

FIG. 21 illustrates a clear partial colocalisation of RNA polymerase II and SPOC1 in the nucleus of DeuserPT cells. The comparison of the expression patterns show concordance of the spatial distribution of SPOC1 and RNA polymerase II. DAPI (A), RNA-Polymerase II (B, E) and SPOC1 (C, F). The overlay of the pictures with anti RNA polymerase II antibody and the polyclonal SPOC1 peptide antibody show the partial colocalisation of the signals in yellow (D). The area marked by a rectangle (D) is shown in a 4-fold amplification for RNA polymerase II (E), SPOC1 (F) and for the overlay of both pictures (G).

Abb. 22: Clear partial colocalisation of transcription factor E2F-1 and SPOC1 in the nucleus of DeuserPT cells. DAPI (A), E2F-1 (B), and SPOC1 (C). The overlay of the pictures with the anti-E2F-1 antibody and the polyclonal SPOC1 peptide antibody shows the significant partial colocalisation of the signals in yellow (D). The rectangle (D) marks a 4-fold amplification of a section for E2F-1 (green), SPOC1 (red) and the overlay of the two pictures (yellow). Examples of further colocalisations are marked with arrows (D).

2. Evolutionary Conservation of SPOC1

A comparison of the amino acid sequence of human SPOC1 and the corresponding sequences of Xenopux and Tetraodon has shown that SPOC1 is highly conserved between these three phylogenetic unrelated species (FIG. 23). Such a conservation is generally observed only for factors playing a crucial role in the physiology of the cell. It is noted that the amino acid sequence for human SPOC1 as indicated in FIG. 23 corresponds to SEQ ID NO: 3 as mentioned herinbefore and in the claims.

FIG. 23: Comparison of the amino acid sequences of human SPOC1 with the corresponding sequences of Xenopus and Tetraodon, as well as the deduction of a consensus sequence. SPOC1 is highly conserved between the three species.

3. Generation of High Affinity Antibodies Against SPOC1

High affinity antibodies against SPOC1 could be generated in both rabbit and chicken egg yolk (FIG. 24). The double bands correspond most probably to different sumolation stages. The availability of SPOC1 specific antibodies will simplify the expression studies for the future.

FIG. 24: Generation and verification of antibodies against SPOC1 raised in rabbit and chicken egg yolk. Western blot analyses of ectopically expressed SPOC1 protein showed that both antibodies detect SPOC1 signals at 36, 40 and 46 kDa in total cell lysates of U2OS-TRex cells.

4. Sumoylation of SPOC1

The SPOC1-protein has a calculated molecular mass of 36.6 kDa. Western blot analyses using the new generated polyclonal rabbit anti-SPOC1 antibody showed a signal for the SPOC1-protein at the expected size but also at approx. 40 and 46 kDa. This aberrant migration behaviour can appear due to covalent modifications of the protein. To identify possible modifications, the SPOC1 amino acid sequence was screened for consensus sites for several covalent modifications.

Analyses of the SPOC1 sequence for consensus sites for SUMO modification (published by Verger et al., EMBO Rep., 2003) revealed two putative sumoylation target sites with high probability at positions 141 and 193 of the SPOC1 amino acid sequence. Further analyses with the SUMOplot Prediction software approved these consensus sites (FIG. 24). The putative sumoylation of SPOC1 could explain the results obtained by western blot analyses. We think that the signal at 36 kDa is the non-sumoylated form of SPOC1, while the signals at 40 and 46 kDa show the mono- and the double-sumoylated forms of SPOC1 respectively.

FIG. 25. Prediction of putative sumoylation target sites in the amino acid sequence of SPOC1 by the SUMOplot prediction software. The two motifs with high probability positions (LKLE and IKTE) are indicated in red.

SEQ ID NO:1

ATGGACTCTGACTCTTGCGCCGCCGCCTTCCACCCGGAGGAATACTCC
CCCAGTTGCGAGAGGCGCAGGACCGTGGAAGACTTCAACAAATTCTGC
ACCTTTGTCTTGGCCTATGCTGGCTACATCCCTTATCCGAAGGAGGAAC
TCCCTTTAAGGAGCAGCCCCAGCCCTGCTAACAGCACTGCTGGTACCA
TTGACAGCGACGGCTGGGACGCGGGTTTCTCAGACATCGCGTCCTCAG
TGCCCTTGCCAGTCTCTGACCGCTGCTTTAGCCACCTGCAGCCTACTCT
CTTGCAGCGAGCCAAGCCCAGTAACTTCCTGCTGGACAGAAAGAAAAC
GGACAAGCTGAAGAAGAAGAAGAAGAGGAAGCGCAGGGACAGTGATG
CGCCTGGGAAAGAGGGGTACAGGGGGGGCTTGCTGAAGCTGGAAGCC
GCTGACCCCTACGTGGAGACCCCCACGAGTCCCACCTTGCAGGATATC
CCCCAGGCTCCCAGCGACCCCTGCTCGGGCTGGGACTCCGATACTCC
CTCGAGTGGATCTTGTGCCACTGTGTCACCTGATCAGGTCAAAGAAATA
AAAACTGAAGGCAAACGGACTATCGTCCGGCAGGGAAAGCAGGTGGT
GTTCCGAGATGAGGACAGCACTGGCAATGATGAGGACATCATGGTGGA
CTCAGATGACGATTCCTGGGACCTCGTGACCTGCTTCTGCATGAAGCC
ATTTGCCGGCCGCCCCATGATCGAGTGTAATGAGTGCCACACCTGGAT
TCACCTGTCCTGTGCGAAAATCCGGAAATCCAATGTTCCAGAAGTGTTT
GTCTGCCAAAAGTGCCGGGACTCCAAGTTTGACATCCGCCGTTCCAAC
CGCTCGCGGACGGGCTCCCGGAAGCTGTTCCTGGACTGA

SEQ ID NO:2

ATGGACTCCGACTCCTGCGCCGCCGCCTTCCACCCCGAGGAGTACTC
CCCCACTTGTAAGAGGCGCCGGACTGTGGAAGACTTCAACAAATTCTG
CACCTTCGTCTTGGCATATGCGGGCTACATCCCCTACCCAAAGGAGGA
GCTCCCCCTGAGGAGCAGTCCCAGCCCCGCCAACAGCACTGCCGGGA
CCATTGACAGCGACGGCTGGGACACTGGTTTCTCTGACATCACGCCTT
CAGTGCCCGACCGATGCTTCAGCCACCTGCAGCCTTCCCTCTTGCAGA
GAGCTAAGCCCAGTAAOTACCTTCTGGACAGGAAGACAACTGACAAGC
TGAAGAAGAAGAAGAGGAGGAAGCGCAGGGACAGCGACATACCTGTG
AAGGAGGGATTCAGGGAGAGCCTGCTGAAGCTGGAAGCTGCAGACCC
ATATGTGGAGACTCCCTCAAGCCCCACCATGCAGGATATTCCCCAGGC
GTCTGCTGACCCCTGCTCAGGCTGGGACTCTGACACACCCTCAAGTGG
CTCTTGTGCTACTGTGTCACCTGATCAGGTCACAGAAATAAAAACTGAA
GGAAAACGGACTATTGTCCGCCAGGGAAAGCAGGTGGTGTTCCGAGAC
GAAGACAGCACTGGCAATGATGAAGACATCATGGTGGACTCAGATGAT
GATTCCTGGGACCTCGTCACCTGTTICTGCATGAAGCCCTTTGCCGGC
CGCCCCATGATCGAGTGTAACGAGTGCCACACCTGGATTCACCTGTCC
TGTGCAAAGATCCGCAAGTCCAATGTCCCGGAAGTTTTTGTCTGCCAAA
AGTGCCGGGACTCCAAGTTTGATATCCGTCGCTCCAACCGGTCCCGAA
TGGGCTCCCGGAAGCTGTTTCTGGACTGA

What is claimed is:

1. Human K203 protein, which is encoded by the nucleic acid of SEQ ID NO: 1 or variants thereof, which variants are each defined as having one or more substitutions, insertions, and/or deletions as compared to the nucleic acid of SEQ ID NO: 1, provided that:
a) these variants hybridize under moderately stringent conditions to a nucleic acid, which comprises the sequence of SEQ ID NO: 1, and further provided that these variants code for a protein having K203 activity; or
b) these variants have nucleic acid changes which are due to the degeneration of the genetic code, which code for the same or functional equivalent amino acid as the nucleic acid of SEQ ID NO: 1.

2. Murine K203 protein, which is encoded by the nucleic acid of SEQ ID NO: 2 or variants thereof, wherein the variants are each defined as having one or more substitutions, insertions and/or deletions as compared to the sequence of SEQ ID NO: 2, provided that:
a) said variants hybridize under moderately stringent conditions to a nucleic acid which comprises the sequence of SEQ ID NO: 2, and further provided that said variants code for a protein having K203 activity; or
b) these variants having nucleic acid changes, which are due to the degeneration of the genetic code, which code for the same or a functional equivalent amino acid as the nucleic acid of SEQ ID NO: 2.

3. An isolated nucleic acid, which comprises the nucleic acid of SEQ ID NO: 1 or variants thereof, wherein the variants are each defined as having one or more substitutions, insertions, and/or deletions as compared to the nucleic acid of SEQ ID NO: 1, provided that:
a) these variants hybridize under moderately stringent conditions to a nucleic acid, which comprises the sequence of SEQ ID NO: 1, and further provided that these variants code for a protein having K203 activity; or
b) said variants have nucleic acid changes which are due to the degeneration of the genetic code, which code for the same or functional equivalent amino acids as the nucleic acid of SEQ ID NO: 1.

4. An isolated nucleic acid which comprises the nucleic acid of SEQ ID NO: 2 or variants thereof, wherein the variants are each defined as having one or more substitutions, insertions, and/or deletions as compared to the sequence of SEQ ID NO: 2, provided that:
a) said variants hybridize under moderately stringent conditions to a nucleic acid, which comprises in the sequence of SEQ ID NO: 2, and further provided that these variants code for a protein having K203 activity; or
b) these variants have nucleic acid changes, which are due to the degeneration of the genetic code, which code for the same or a functional equivalent amino acid as compared to the nucleic acid of SEQ ID NO: 2.

5. The isolated nucleic acid of claim 3 or 4, which is further operably linked to one or more regulatory sequences.

6. A nucleic ac:id, which is a transcriptional product of one of the nucleic acids of claims 3 or 4, preferably mRNA or siRNA.

7. A nucleic acid, which selectively hybridizes to transcriptional products of claim 6 under moderately stringent conditions.

8. The nucleic acid of claim 7, which is antisense DNA or RNA.

9. A DNA- or RNA-probe which hybridizes to one of the nucleic acids of claim 3 or 4.

10. A vector, which comprises one of the nucleic acids of claims 3 or 4.

11. An expression vector, which comprises the nucleic acid sequence of claims 3 or 4 and one or more regulatory sequences.

12. The vector of claim 11 which is a plasmid.

13. A host cell, which has been transformed with the vector of claim 11 or 12.

14. The host cell of claim 13, which is a eucaryotic cell.

15. The host cell of claim 14, which is a mammalian cell, plant cell, yeast cell, or an insect cell.

16. The mammalian cell of claim 15, which is a CHO—, COS—, HeLa—, 293T-, HEH—, or BHK-cell.

17. The mammalian host cell of claim 15, which is an adult or embryonic stem cell.

18. The host cell of claim 13, which is a procaryotic cell.

19. The host cell of claim 18, which is E.coli or Bacillus subtilis.

20. A binding compound, preferably an antibody, small molecule or an aptamer, or K203 binding peptide, spiegelmere, aptazyme, and ribozyme which is directed against the K203 protein of claim 1, 2 or 35.

21. The antibody of claim 20, wherein said antibody is selected from the group consisting of a polyclonal antibody, a monoclonal antibody, a humanized antibody, a chimeric antibody, and a synthetic antibody.

22. The antibody of claim 21, which is linked to a toxic agent, and/or to a detectable agent.

23. A hybridoma, which produces a monoclonal antibody having binding specificity for the K203 proteins of claims 1, 2 or 35.

24. A pharmaceutical composition, comprising a therapeutically effective dose of a nucleic acid of claim 8 in combination with a pharmaceutically acceptable carrier.

25. A pharmaceutical composition, comprising a therapeutically effective dose of an antibody or aptamer of claim 20-22 or a compound of claim 37 in combination with a pharmaceutically acceptable carrier.

26. A diagnostic composition, comprising a K203 binding compound, preferably an antibody, small molecule or an aptamer, of claim 20-22.

27. A diagnostic composition, comprising the probe of claim 9.

28. A transgenic mouse in which the nucleic acid of claim 4 has been inactivated.

29. A transgenic non-human mammal, in the genome of which a nucleic acid of claim 3 or 4 has been inserted.

30. An ex-vivo method for the diagnosis of cancer comprising the following steps:
a) providing a tissue sample or a serum sample from a patient;
b) qualitative and/or quantitative determination of the transcriptional products of claim 6 or of the K203 protein of claim 1 in the sample; wherein
an overexpression of the transcriptional products of claim 6 or of the K203 protein of claim 1 in the tissue or serum sample is indicative for the presence of cancer and the degree of expression is indicative for the prognosis of said patient.

31. The method of claim 30, wherein the determination in step b) is performed by Northern Blot, in situ hybridization or RT-PCR, preferably semiquantitative RT-PCR, or a combination thereof.

32. The method of claim 30, wherein the determination in step b) is performed by using a composition of claim 26 or 27.

33. A method of treating cancer, comprising administering an therapeutically effective amount of the pharmaceutical composition of claim 24 or 25 to a patient in need of such treatment.

34. The method of of claim 30 or 33, wherein the cancer is selected from ovarian cancer, chondrosarcoma, osteosarcoma, neuroblastoma, endometrial cancer, cervix cancer, germ cell tumors, thyroid cancer, lung cancer, prostate cancer, colon cancer, kidney cancer, bladder cancer, esophageal cancer, rectal cancer, meningioma and other tumors of the central nervous system, parathyroid cancer, hepatocellular cancer and hematological malignancies.

35. A human K203 protein having the amino acid sequence of SEQ ID NO: 3 or a variant of said sequence, wherein said variant comprises one or more insertions, substitutions and/or deletions as compared to the sequence of SEQ ID NO: 3, and wherein the biological activity is substantially equal to the activity of the protein comprising the unmodified amino acid sequence of SEQ ID NO: 3.

36. A screening method for identifying an antagonist capable of inhibiting or blocking the K203 protein of claim 1, 2 or 35, comprising the steps of:
(a) generating or providing mammalian K203,
(b) contacting said K203 with a candidate compound,
(c) detecting the inhibition or blocking of said compound by a suitable detection method,
(d) selecting a compound that has been tested positive in step (c),
(e) optionally repeating steps (a)-(d) with a suitably modified form of the compound of step (d).

37. A compound, which is capable of inhibiting or blocking the K203 protein of claim 1, 2 or 35 and/or which is obtainable by the method of claim 36.


SEQUENCE LISTING (5 sequences)

SEQ ID NO: 1 - DNA, 903 bp, Homo sapiens
atggactctgactcttgcgccgccgccttccacccggaggaatactcccccagttgcgagaggcgcaggaccgtggaagacttcaacaaattctgcacctttgtcttggcctatgctggctacatcccttatccgaaggaggaactccctttaaggagcagccccagccctgctaacagcactgctggtaccattgacagcgacggctgggacgcgggtttctcagacatcgcgtcctcagtgcccttgccagtctctgaccgctgctttagccacctgcagcctactctcttgcagcgagccaagcccagtaacttcctgctggacagaaagaaaacggacaagctgaagaagaagaagaagaggaagcgcagggacagtgatgcgcctgggaaagaggggtacagggggggcttgctgaagctggaagccgctgacccctacgtggagacccccacgagtcccaccttgcaggatatcccccaggctcccagcgacccctgctcgggctgggactccgatactccctcgagtggatcttgtgccactgtgtcacctgatcaggtcaaagaaataaaaactgaaggcaaacggactatcgtccggcagggaaagcaggtggtgttccgagatgaggacagcactggcaatgatgaggacatcatggtggactcagatgacgattcctgggacctcgtgacctgcttctgcatgaagccatttgccggccgccccatgatcgagtgtaatgagtgccacacctggattcacctgtcctgtgcgaaaatccggaaatccaatgttccagaagtgtttgtctgccaaaagtgccgggactccaagtttgacatccgccgttccaaccgctcgcggacgggctcccggaagctgttcctggactga

SEQ ID NO: 2 - DNA, 891 bp, Mus sp.
atggactccgactcctgcgccgccgccttccaccccgaggagtactcccccacttgtaagaggcgccggactgtggaagacttcaacaaattctgcaccttcgtcttggcatatgcgggctacatcccctacccaaaggaggagctccccctgaggagcagtcccagccccgccaacagcactgccgggaccattgacagcgacggctgggacactggtttctctgacatcacgccttcagtgcccgaccgatgcttcagccacctgcagccttccctcttgcagagagctaagcccagtaactaccttctggacaggaagacaactgacaagctgaagaagaagaagaggaggaagcgcagggacagcgacatacctgtgaaggagggattcagggagagcctgctgaagctggaagctgcagacccatatgtggagactccctcaagccccaccatgcaggatattccccaggcgtctgctgacccctgctcaggctgggactctgacacaccctcaagtggctcttgtgctactgtgtcacctgatcaggtcacagaaataaaaactgaaggaaaacggactattgtccgccagggaaagcaggtggtgttccgagacgaagacagcactggcaatgatgaagacatcatggtggactcagatgatgattcctgggacctcgtcacctgtttctgcatgaagccctttgccggccgccccatgatcgagtgtaacgagtgccacacctggattcacctgtcctgtgcaaagatccgcaagtccaatgtcccggaagtttttgtctgccaaaagtgccgggactccaagtttgatatccgtcgctccaaccggtcccgaatgggctcccggaagctgtttctggactga

SEQ ID NO: 3 - protein, 300 aa, Homo sapiens
MDSDSCAAAFHPEEYSPSCERRRTVEDFNKFCTFVLAYAGYIPYPKEELPLRSSPSPANSTAGTIDSDGWDAGFSDIASSVPLPVSDRCFSHLQPTLLQRAKPSNFLLDRKKTDKLKKKKKRKRRDSDAPGKEGYRGGLLRLEAADPYVETPTSPTLQDIPQAPSDPCSGWDSDTPSSGSCATVSPDQVKEIRTEGKRTIVRQGKQVVFRDEDSTGNDEDIMVDSDDDSWDLVTCFCMKPFAGRPMIECNECHTWIHLSCAKIRKSNVPEVFVCQKCRDSKFDIRRSNRSRTGSRKLFLD

SEQ ID NO: 4 - protein, 325 aa, Xenopus sp.
MDTASTSTFDSEDGTPNYRRRRTVEDFNKFCTFVLAYAGYIPYPEEETPLRASSSPPNSTGSTVDSDSWDSRYQDLPSSDALPIKKRRSFSDLKHERSFPTVLKRSKLSSFLLDRKKIDKIKKKKKLKKKELELEGIKQEVEPVKPIIKQEPVETPIEETTPISPIAPFPDDHLELKKSPPHEIQEPVLEDPSTSMDCPSVSMSYEDTEDIKTEYSEVNLGKRTVIRQGKQVVFRDEDSTGNDEDIMVDSDDESWHLVTCFCMKPFAGRPMIECNECNTWIHLSCAKIRKSNVPEIYVCQKCRDTKFDIRRSNRSRMGCRKLFLD

SEQ ID NO: 5 - protein, 291 aa, Tetraodon
KRRRTVEDFNKFCTFVLAYAGYIPYPQEDSPLRSSSSPPNSTGSTADSDGWTPGPPTSCPQRPPKVPHDRTRKHPQSGGLLSSHTKKEANERRKADKLKKKKRRKERQPSAEEDTLVPEFQHPISFRGQTIKEEPEDEISIPLHPQRLLSPVPCKEEPSEGQQNWDGQDLKEGVVRVQKVEGFSGNRTVFRQGKQVVFRDEDGSGEDEDIMVDSDDDSWDLVTCFCMKPFAGRAMIECNKCHTWIHLSCAKIRKSHVPETYVCQRCRETQGLQDTRRSHRSRAVPSKHTLD